(19) United States
(12) Patent Application Publication
(10) Pub. No.: US 2026/0146244 A1
(43) Pub. Date: May 28, 2026

(54) MATERIALS AND METHODS FOR THE PRODUCTION OF PHENYLALANINE

(71) Applicant: Purdue Research Foundation, West Lafayette, IN (US)

(72) Inventors: John A. Morgan, West Lafayette, IN (US); Arnav Deshpande, Broomfield, CO (US); Melissa Marsing, West Lafayette, IN (US)

(73) Assignee: Purdue Research Foundation, West Lafayette, IN (US)

(21) Appl. No.: 19/373,388

(22) Filed: Oct. 29, 2025

Related U.S. Application Data
(60) Provisional application No. 63/724,627, filed on Nov. 25, 2024.

(51) Int. Cl. C12N 9/88; C07K 14/195; C12N 15/74; C12P 13/22; C12R 1/01

(52) U.S. Cl. CPC C12N 9/88; C07K 14/195; C12N 15/74; C12P 13/222; C12R 2001/01; C12Y 402/01051

(57) ABSTRACT

Polynucleotides, constructs/vectors, host cells, polypeptides, and methods of producing phenylalanine in a prokaryotic host, such as for subsequent isolation.

### CROSS REFERENCE TO RELATED APPLICATIONS

This application claims priority to U.S. provisional patent application No. 63/724,627, which was filed Nov. 25, 2024, and which is hereby incorporated by reference in its entirety.

### TECHNICAL FIELD

The present disclosure relates to nucleic acids, vectors, host cells, polypeptides, and methods of producing phenylalanine in a prokaryotic host, such as a cyanobacterium.

### SEQUENCE LISTING

A computer-readable form (CRF) of the Sequence Listing is submitted with this application. The sequence listing is entitled 70934-02_SEQ_LISTING.xml, was generated on Oct. 17, 2025, and is 8389 bytes in size. The entire content of the sequence listing is incorporated herein by reference in its entirety.

### BACKGROUND

Cyanobacteria are promising organisms for metabolic engineering due to their ability to use sunlight and CO₂ to produce biochemicals and biofuels sustainably¹. However, industrial use of cyanobacteria requires addressing several barriers such as, but not limited to, economical scale up and improving product titers². Improving the rate of carbon fixation, and thus the production of growth-associated biochemicals, can increase the feasibility of using cyanobacteria in industrial applications³. Approaches to improve carbon fixation and photosynthetic efficiency in have thus far been directed at engineering of ribulose-1,5-bisphosphate carboxylase/oxygenase (RuBisCO) enzyme^{4,5}, enabling greater light penetration by reduction of antenna size⁶, manipulation of the Calvin-Benson-Bassham (CBB) cycle⁵, engineering of photorespiratory bypass mechanisms^{7,8}, and reducing dissipative losses⁹.

Recently, engineering of the heterologous production of biochemicals (“sink engineering”) in cyanobacteria has been shown to produce a significant augmentation of carbon fixation¹⁰. Various cyanobacteria engineered to produce biochemicals such as sucrosen,^{11,12}, 2,3-butanediol (2,3 BD)¹³, 2-phenylethanol¹⁴, glycerol¹⁵, phenylpropanoids¹⁶, and ethylene¹⁷ have shown increased carbon fixation ability. However, neither the underlying mechanism of enhanced C-fixation nor the effect of light intensity on carbon assimilation is well understood. Investigating the dynamics of this source-sink relationship is particularly important for evaluating and improving the feasibility of cyanobacteria in outdoor cultivation applications in which light intensities can vary substantially.

It has been previously hypothesized that under excess light, introduction of a heterologous sink can improve the utilization of the excess light energy¹⁸. However, it was recently shown that a sucrose sink showed highest improvement in photosynthetic flux at low light, whereas under high light, an electron sink, cytochrome P450, led to photosynthetic enhancement¹². Furthermore, the extent of the increase in electron transport in both cases was substantially lower compared to the enhancement observed in carbon fixation¹².

Introduction of a heterologous sink or biomass composition can result in a change of adenosine triphosphate (ATP) to nicotinamide adenine dinucleotide phosphate (NADPH) demand, which is an important factor in photosynthetic productivity¹⁹⁻²¹. Linear electron flow through photosystem II (PSII) and photosystem I (PSI) in S. elongatus generates an ATP/NADPH demand of 1.38, due to the presence of 13 c rings in ATP synthase²², whereas the CBB cycle requires an ATP/NADPH ratio of 1.5²³. Alternate electron flow (AEF), one of which is cyclic electron flow (CEF) around PSI, produces only ATP and can make up the shortfall in ATP^{24,25}. However, the effect of additional sinks on linear electron flow (LEF) and CEF under different light conditions remains unclear in cyanobacteria.

### SUMMARY

Provided is an isolated or purified polynucleotide encoding a prephenate dehydratase having an amino acid sequence that is at least about 90% identical to the amino acid sequence of SEQ ID NO: 1:
1 MAALRFQAWL TDQDQQPSHL LACRSIPATL QTLADGTVDY AVVPVENSVE GSVAATLDSL
61 WQLPQLSIQR ALILPIAHAL ISFEDDRTAI RQVLSHPQAL AQCQQWLQRQ LPQAELIPTN
121 STTEALQDLE RHPQRAVIAS TRAAELYQMP IQSFPINDSP DNRTRFWVVS RNLTPGGACT
181 SLSFSLDANV PGALVKPLQI LAERQINLSR IESRPTKRSL [symbol] EYLFFLDLE ADLREPAIAQ
241 AVQAVADCTE QLRVLGSYDS LDFTQVVQPS [SEQ ID NO: 1], in which the glycine at amino acid position 221 (G221) is substituted with an amino acid having an aromatic R group. G221 can be substituted with an amino acid selected from phenylalanine, tyrosine, and tryptophan. G221 can be substituted with tryptophan (G221W) [SEQ ID NO: 4].

Further provided is an isolated or purified polynucleotide encoding a TolC family protein having an amino acid sequence that is at least about 90% identical to the amino acid sequence of SEQ ID NO: 2:
1 MAAFLYRLSL LSALAIAAHG MEPPAAIADI VDPQATGPSP TIAQNSPPPA ATTPAPTTPP
61 SSPVKEVVPD ANLLKELQAN PNPFQLPNQP NQVQTESLQP LTLEQALNLA RLNNPQVQIR
121 QLQVEQRRAA LRGTEAALYP TLGLQGNAGY QQSGNRLNVT EGSTVQPTGS SLFTTLGQSS
181 IGATLNLNYT IFDFVRGAQL AASRDQVTQA ELDLEATLED LQLTVSEAYY QLQNADQLVR
241 IARESVVASE RSLKDAEALF RAGVGTQFDV LRQQVQLAQD QQNLVDSIGN QDKARRSLVQ
301 ALNLPQNVNV LTADPVELAA PWNLSLDESI VLAFQNRPEL EREVLQRNIS YNQAQAARGQ
361 ILPQLGLQAS YGVTGSINSN LRSGSQALTF PSPTLTNNSN YSYSIGLVLN VPLFDGGLAN
421 ANAQQQELNG QIAEQNFVLT RNQIRTDVET AFYDLQTNLA NIGTTRKAVE QAREALRLAR
481 LRFQAGVGTQ TEVIDSQRDL TRAEANALNA ITAYNLALAR IKRAVSNVSN [symbol] ARVGS [SEQ ID NO: 2], in which the leucine at amino acid position 531 (L531) is substituted with an amino acid having an aromatic R group. L531 can be substituted with an amino acid selected from phenylalanine, tyrosine, and tryptophan. L531 can be substituted with tryptophan (L531W) [SEQ ID NO: 5].

Still further provided is an isolated or purified polynucleotide encoding a circadian clock protein having an amino acid sequence that is at least about 90% identical to the amino acid sequence of SEQ ID NO: 3:
1 MLSQIAICIW VESTAILQDC QQALAGDRYQ LQICSSGDLL LDYAQTHRDQ IDCLLLVATN
61 PGCKTVIQQL CFQGIVVPAI VVGDRDTDDV TDTQKDWVYH SAELHLGIHQ LEQLPYQVDA
121 ALAEFLRQAP VETIADQVML MAATHDPELA SHQRDLAQRL QERLGYLGVY YKRDPDRFLR
181 NLPA [symbol] EGQKL LEAMQTSYRE IVLSYFSPNS NLNQSLDNFV NMAFFADVPV TQVVEIHMEL
241 MDEFAKKLRV EGRSEDILLD YRLTLIDVIA HLCEMYRRSI PRET [SEQ ID NO: 3], in which there is a frameshift mutation at tyrosine at amino acid position 185 (Y185) resulting in the substitution of Y185 with an amino acid having a nonpolar, aliphatic R group. Y185 can be substituted with an amino acid sequence selected from glycine, alanine, valine, leucine, methionine, or isoleucine. Y185 can be substituted with leucine (Y185L) [SEQ ID NO: 6].

A construct or vector comprising an above-described isolated or purified polynucleotide operably linked to a promoter is also provided. A host cell comprising and expressing the construct or vector is further provided. In embodiments, the host cell comprises and expresses the isolated or purified polynucleotide encoding a prephenate dehydratase having an amino acid sequence that is at least about 90% identical to the amino acid sequence of SEQ ID NO: 1, in which the glycine at amino acid position 221 (G221) is substituted with tryptophan (G221W) [SEQ ID NO: 4]. In embodiments, the host cell comprises and expresses the isolated or purified polynucleotide encoding a TolC family protein having an amino acid sequence that is at least about 90% identical to the amino acid sequence of SEQ ID NO: 2, in which the leucine at amino acid position 531 is substituted with tryptophan (L531W) [SEQ ID NO: 5]. In embodiments, the host cell comprises and expresses the isolated or purified polynucleotide encoding a circadian clock protein having an amino acid sequence that is at least about 90% identical to the amino acid of SEQ ID NO: 3, in which the tyrosine at amino acid position 185 is substituted with leucine (Y185L) [SEQ ID NO: 6]. In embodiments, the host cell comprises and expresses the isolated or purified polynucleotide encoding a prephenate dehydratase having an amino acid sequence that is at least about 90% identical to the amino acid sequence of SEQ ID NO: 1, in which the glycine at amino acid position 221 (G221) is substituted with tryptophan (G221W) [SEQ ID NO: 4], and the isolated or purified polynucleotide encoding a TolC family protein having an amino acid sequence that is at least about 90% identical to the amino acid sequence of SEQ ID NO: 2, in which the leucine at amino acid position 531 is substituted with tryptophan (L531W) [SEQ ID NO: 5]. In embodiments thereof, the host cell further comprises and expresses the isolated or purified polynucleotide encoding a circadian clock protein having an amino acid sequence that is at least about 90% identical to the amino acid of SEQ ID NO: 3, in which the tyrosine at amino acid position 185 is substituted with leucine (Y185L) [SEQ ID NO: 6]. The host cell can be a Cyanobacterium.

Still further provided is an isolated or purified polypeptide having the amino acid sequence of SEQ ID NO: 4, 5, or 6.

Even still further provided is a method of producing phenylalanine in a prokaryotic host. The method comprises culturing an above-described host cell in medium under culture conditions that support the production of phenylalanine. The method can further comprise isolating the phenylalanine, e.g., by collecting phenylalanine from a biomass of the cultured prokaryotic host or a culture medium thereof. In embodiments, the prokaryotic host is Cyanobacterium, and the medium is modified BG-11. In embodiments, the BG-11 medium is supplemented with concentrated modified BG-11 medium about every three days.

### FIGURES

FIG. 1. Schematic representation of cyanobacterial photosynthetic machinery showing electron transfer pathways, non-photochemical quenching pathway, action of electron transport inhibitors, Calvin cycle, biomass and Phe sinks, and ATP/NADPH synthesis and consumption.
† can vary depending on nitrogen source, carbon uptake, amino acid re-uptake, carbon recycling, etc.
The NPQ arrows indicate loss of electrons to photoprotective mechanisms.
Abbreviations: photosystem II (PSII), photosystem I (PSI), plastoquinone (PQ), plastocyanin (PC), cytochrome b₆f (Cyt b₆f), linear electron flow (LEF), cyclic electron flow (CEF), non-photochemical quenching (NPQ), Calvin Benson Bassham cycle (CBB), phenylalanine (Phe), phosphoenolpyruvate (PEP), erythrose 4-phosphate (E4P), hydroxylamine (HA), 3-(3,4-dichlorophenyl)-1,1-dimethylurea (DCMU), dibromothymoquinone (DBMIB), ferridoxin NADP⁺ oxidoreductase (FNR), and ferridoxin (Fd).

FIG. 2A. Phe titer of wild-type and mutants when cultured for 3 days at 240 μmol m⁻²s⁻¹ under 3% CO₂.

FIG. 2B. Total capacity of biomass and Phe sinks. * indicates p<0.05 using a two-tailed two-sample t-test.

FIG. 3. Distribution of fixed carbon in biomass, Phe, and the total sink (biomass+Phe) in WT, M14, and M14.2 under LL (40 μmol photons m⁻² s⁻¹) (A-C), ML (240 μmol photons m⁻² s⁻¹) (D-F), and HL (1000 μmol photons m⁻² s⁻¹) (G-I) conditions. Data represent mean and standard deviation of three biological replicates inoculated at the same density and cultured at 38° C. and ambient CO₂. The x-axis represents time in hours, and the y-axis represents carbon content in mg/L.

FIGS. 4A-4C show Phe-overproducing strains assimilate more carbon. (FIG. 4A) Ratio of total carbon fixed by M14 and M14.2 to WT at the end of two days. Statistical comparison was performed with a one tailed t-test with the null hypothesis mean ≤1. (FIG. 4B and FIG. 4C) Percentage of carbon that is diverted to the Phe sink under different light conditions in M14 and M14.2, respectively. Data represent mean and standard deviation of three biological replicates inoculated at the same density and cultured at 38° C. and ambient CO₂.

FIGS. 5A-5B show M14.2 shows higher PSII operating efficiency and linear electron transport. (FIG. 5A) Effective quantum yield of PSII and (FIG. 5B) relative electron transfer rate through PSII (linear electron flow) under different light intensities in WT and M14.2. Data are the mean and standard deviations of three biological replicates.

FIGS. 6A-6F show PSI redox kinetics of WT and M14.2. Light-induced oxidation kinetics of P700 in LL- (FIG. 6A), ML- (FIG. 6B), and HL- (FIG. 6C) acclimated strains. For each growth light, a corresponding actinic light of comparable intensity was used for determination of the intermediate P700 oxidation state P. Traces for untreated, DCMU and HA, and DBMIB-treated samples are given. The dark re-reduction kinetics of P700 (LL is FIG. 6D; ML is FIG. 6E, and HL is FIG. 6F) are shown after normalization to Pₘ′ as described previously⁴¹. Traces are representatives of three biological replicates.

FIGS. 7A-7C show partitioning of electrons into linear and cyclic pathways in WT and M14.2. (FIG. 7A) Estimation of the fraction of cyclic electron flow in WT and M14.2 under different light intensities. (FIG. 7B) rETR1 and the contribution of linear and cyclic flow towards it. (FIG. 7C) ΦPSI is plotted against ΦPSII to determine the extent of cyclic and linear electron flow. Data represent mean and standard deviation from three biological replicates. Statistical significance is calculated using a two tailed t-test.

FIGS. 8A-8C show the effect of Phe sink on PSI photochemical efficiency. (FIG. 8A) Photochemical efficiency of PSI (ΦPSI) at LL, ML, and HL in WT and M14.2. The nonphotochemical energy dissipation due to (FIG. 8B) donor side limitation Y(ND) and (FIG. 8C) acceptor side limitation Y(NA). Data represent mean and standard deviation of three biological replicates.

### DETAILED DESCRIPTION

Disclosed is the identification of single nucleotide polymorphisms (SNPs) underlying phenylalanine (Phe) overproduction in Phe-overproducing strains generated by random mutagenesis of the fast-growing Cyanobacterium S. elongatus PCC 11801²⁶ and selection on Phe analogues as described in USPAPN 2023/0013336, published Jan. 19, 2023. Mutants with the Phe sink showed increased net carbon fixation. Carbon fixation can be improved using heterologous and homologous (i.e., endogenous products) sinks. The sink-derived improvement in carbon fixation is light-dependent. The ratio of adenosine diphosphate (ADP)/nicotinamide adenine dinucleotide phosphate (NADPH) can be altered. FIG. 1 is a schematic diagram of cyanobacterial photosynthetic machinery, electron transport pathways, Phe sink, ATP and NADPH production and demands, and the site of action of photosynthetic inhibitors.

In view of the above, provided is an isolated or purified polynucleotide encoding a prephenate dehydratase having an amino acid sequence that is at least about 90% (or at least 95%, 96%, 97%, 98% or 99%) identical to the amino acid sequence of SEQ ID NO: 1:
1 MAALRFQAWL TDQDQQPSHL LACRSIPATL QTLADGTVDY AVVPVENSVE GSVAATLDSL
61 WQLPQLSIQR ALILPIAHAL ISFEDDRTAI RQVLSHPQAL AQCQQWLQRQ LPQAELIPTN
121 STTEALQDLE RHPQRAVIAS TRAAELYQMP IQSFPINDSP DNRTRFWVVS RNLTPGGACT
181 SLSFSLDANV PGALVKPLQI LAERQINLSR IESRPTKRSL [symbol] EYLFFLDLE ADLREPAIAQ
241 AVQAVADCTE QLRVLGSYDS LDFTQVVQPS [SEQ ID NO: 1], in which the glycine at amino acid position 221 (G221) is substituted with an amino acid having an aromatic R group. G221 can be substituted with an amino acid selected from phenylalanine, tyrosine, and tryptophan. G221 can be substituted with tryptophan (G221W) [SEQ ID NO: 4]. In various embodiments, prephenate dehydratase, along with chorismate mutase, is encoded by the pheA gene as a bifunctional enzyme. Since prephenate dehydratase and chorismate mutase are subject to feedback inhibition by L-phenylalanine, genes encoding these enzymes that include a mutation for desensitization to feedback inhibition may be used for enhancing the activities of these enzymes.

Further provided is an isolated or purified polynucleotide encoding a TolC family protein having an amino acid sequence that is at least about 90% (or at least 95%, 96%, 97%, 98% or 99%) identical to the amino acid sequence of SEQ ID NO: 2:
1 MAAFLYRLSL LSALAIAAHG MEPPAAIADI VDPQATGPSP TIAQNSPPPA ATTPAPTTPP
61 SSPVKEVVPD ANLLKELQAN PNPFQLPNQP NQVQTESLQP LTLEQALNLA RLNNPQVQIR
121 QLQVEQRRAA LRGTEAALYP TLGLQGNAGY QQSGNRLNVT EGSTVQPTGS SLFTTLGQSS
181 IGATLNLNYT IFDFVRGAQL AASRDQVTQA ELDLEATLED LQLTVSEAYY QLQNADQLVR
241 IARESVVASE RSLKDAEALF RAGVGTQFDV LRQQVQLAQD QQNLVDSIGN QDKARRSLVQ
301 ALNLPQNVNV LTADPVELAA PWNLSLDESI VLAFQNRPEL EREVLQRNIS YNQAQAARGQ
361 ILPQLGLQAS YGVTGSINSN LRSGSQALTF PSPTLTNNSN YSYSIGLVLN VPLFDGGLAN
421 ANAQQQELNG QIAEQNFVLT RNQIRTDVET AFYDLQTNLA NIGTTRKAVE QAREALRLAR
481 LRFQAGVGTQ TEVIDSQRDL TRAEANALNA ITAYNLALAR IKRAVSNVSN [symbol] ARVGS [SEQ ID NO: 2], in which the leucine at amino acid position (L531) is substituted with an amino acid having an aromatic R group. L531 can be substituted with an amino acid selected from phenylalanine, tyrosine, and tryptophan. L531 can be substituted with tryptophan (L531W) [SEQ ID NO: 5].

Still further provided is an isolated or purified polynucleotide encoding a circadian clock protein having an amino acid sequence that is at least about 90% (or at least 95%, 96%, 97%, 98% or 99%) identical to the amino acid sequence of SEQ ID NO: 3:
1 MLSQIAICIW VESTAILQDC QQALAGDRYQ LQICSSGDLL LDYAQTHRDQ IDCLLLVATN
61 PGCKTVIQQL CFQGIVVPAI VVGDRDTDDV TDTQKDWVYH SAELHLGIHQ LEQLPYQVDA
121 ALAEFLRQAP VETIADQVML MAATHDPELA SHQRDLAQRL QERLGYLGVY YKRDPDRFLR
181 NLPA [symbol] EGQKL LEAMQTSYRE IVLSYFSPNS NLNQSLDNFV NMAFFADVPV TQVVEIHMEL
241 MDEFAKKLRV EGRSEDILLD YRLTLIDVIA HLCEMYRRSI PRET [SEQ ID NO: 3], in which there is a frameshift mutation at tyrosine at amino acid position 185 (Y185) resulting in the substitution of Y185 with an amino acid having a nonpolar, aliphatic R group. Y185 can be substituted with an amino acid sequence selected from glycine, alanine, valine, leucine, methionine, or isoleucine. Y185 can be substituted with leucine (Y185L) [SEQ ID NO: 6].

The term “polynucleotide” may be used interchangeably with “polynucleotide sequence,” “nucleotide sequence,” “nucleic acid sequence,” “nucleic acid,” and “nucleic acid molecule.” Such terms may be used to refer to a nucleotide, an oligonucleotide, a polynucleotide, or any fragment thereof. These phrases also may be used to refer to DNA or RNA of natural or synthetic origin (which may be single-stranded or double-stranded and may represent the sense or the antisense strand). The polynucleotides may be cDNA or genomic DNA.

The phrases “% sequence identity,” “percent identity,” and “% identity” refer to the percentage of residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. A suite of commonly used and freely available sequence comparison algorithms is provided by the National Center for Biotechnology Information (NCBI). The Basic Local Alignment Search Tool (BLAST® alignment tool) is available from several sources, including the NCBI. The BLAST® alignment tool software suite includes various sequence analysis programs including “blastp,” which is used to align a known amino acid sequence with other amino acids sequences from a variety of databases.

Homologous nucleic acids are contemplated. Those of ordinary skill in the art understand the degeneracy of the genetic code and that a variety of polynucleotides can encode the same polypeptide. In some embodiments, the polynucleotides may be codon-optimized for expression in a particular cell including, without limitation, a bacterial cell, a plant cell, or a fungal cell. Thus, non-naturally occurring sequences may be used. These may be desirable, for example, to enhance expression in heterologous expression systems of polypeptides or proteins. Computer programs for generating degenerate coding sequences are available and can be used for this purpose. Degenerate coding sequences can also be generated manually.

A construct or vector comprising an above-described isolated or purified polynucleotide operably linked to a promoter is also provided. The term “construct” refers to recombinant polynucleotides including, without limitation, DNA and RNA, which may be single-stranded or double-stranded and may represent the sense or the antisense strand. The term “vector” refers to a polynucleotide that can transport another polynucleotide to which it has been linked. In some embodiments, the vector may be a “plasmid,” which refers to a circular double-stranded DNA loop into which additional DNA segments may be ligated. Certain vectors are capable of autonomous replication in a host cell into which they are introduced (e.g., bacterial vectors having a bacterial origin of replication and episomal mammalian vectors). Other vectors can be integrated into the genome of a host cell upon introduction into the host cell, where they are replicated along with the host genome, such as some viral vectors or transposons. Plant mini chromosomes are also included as vectors. Vectors may carry genetic elements, such as those that confer resistance to certain drugs or chemicals. Constructs may also include recombinant polynucleotides created using, for example, recombinant DNA methodologies. The term “vector” may be used interchangeably with “construct.” Recombinant polynucleotides include polynucleotide sequences derived from at least two different natural sources, or they may be synthetic. Constructs may include new modifications to endogenous genes introduced by, for example, genome editing technologies. The constructs provided herein may be prepared by methods available to those of skill in the art. Generally, the nomenclature and the laboratory procedures utilized include molecular, biochemical, and recombinant DNA techniques that are well known and commonly employed in the art. Standard techniques available to those skilled in the art may be used for cloning, DNA and RNA isolation, amplification and purification. Such techniques are thoroughly explained in the literature.

The promoter may be a heterologous promoter or an endogenous promoter. The terms “promoter,” “heterologous promoter,” “endogenous promoter,” “promoter region,” and “promoter sequence” may be used to refer generally to transcriptional regulatory regions of a gene. A polynucleotide is “operably connected” or “operably linked” when it is placed into a functional relationship with a second polynucleotide sequence, such as a promoter, which can effect transcription of the polynucleotide. In various embodiments, the polynucleotide may be operably linked to at least 1, at least 2, at least 3, at least 4, at least 5, or at least 10 promoters. A pho regulon promoter can be used for expression in a bacterial host such as Enterobacteriaceae (e.g., Escherichia, Enterobacter, Pantoea, Klebsiella, and Serratia (see, for example, USPAPN 2010/0184162, published Jul. 22, 2010).

Heterologous promoters include, but are not limited to, constitutive, inducible, temporally regulated, developmentally regulated, chemically regulated, tissue-preferred and tissue-specific promoters. The heterologous promoter may be a bacterial, fungal, synthetic, plant, or animal promoter. Those of skill in the art are familiar with a wide variety of additional promoters for use in various cell types.

A host cell comprising and expressing the construct or vector is further provided. Any suitable cell can be used as a host cell. Desirably, the cell produces or can be modified to produce, or to produce at an increased level compared to a wild-type cell, phenylalanine. Suitable “cells” that may be used include eukaryotic or prokaryotic cells. Suitable prokaryotic cells include, without limitation, gram-negative and gram-positive bacterial species. Specific examples of bacterial include Cyanobacterium and Escherichia coli (see, e.g., USPAPN 2012/0219995, published Aug. 30, 2012, paragraph [0075], which is specifically incorporated herein by reference for its teachings regarding same). Suitable eukaryotic cells include, without limitation, plant cells, fungal cells, and animal cells. In embodiments, the host cell comprises and expresses the isolated or purified polynucleotide encoding a prephenate dehydratase having an amino acid sequence that is at least about 90% identical to the amino acid sequence of SEQ ID NO: 1, in which the glycine at amino acid position 221 (G221) is substituted with tryptophan (G221W) [SEQ ID NO: 4]. In embodiments, the host cell comprises and expresses the isolated or purified polynucleotide encoding a TolC family protein having an amino acid sequence that is at least about 90% identical to the amino acid sequence of SEQ ID NO: 2, in which the leucine at amino acid position 531 is substituted with tryptophan (L531W) [SEQ ID NO: 5]. In embodiments, the host cell comprises and expresses the isolated or purified polynucleotide encoding a circadian clock protein having an amino acid sequence that is at least about 90% identical to the amino acid of SEQ ID NO: 3, in which the tyrosine at amino acid position 185 is substituted with leucine (Y185L) [SEQ ID NO: 6]. In embodiments, the host cell comprises and expresses the isolated or purified polynucleotide encoding a prephenate dehydratase having an amino acid sequence that is at least about 90% identical to the amino acid sequence of SEQ ID NO: 1, in which the glycine at amino acid position 221 (G221) is substituted with tryptophan (G221W) [SEQ ID NO: 4], and the isolated or purified polynucleotide encoding a TolC family protein having an amino acid sequence that is at least about 90% identical to the amino acid sequence of SEQ ID NO: 2, in which the leucine at amino acid position 531 is substituted with tryptophan (L531W) [SEQ ID NO: 5]. In embodiments thereof, the host cell further comprises and expresses the isolated or purified polynucleotide encoding a circadian clock protein having an amino acid sequence that is at least about 90% identical to the amino acid of SEQ ID NO: 3, in which the tyrosine at amino acid position 185 is substituted with leucine (Y185L) [SEQ ID NO: 6]. The host cell can be a Cyanobacterium.

The construct or vector can be introduced into a host cell by any suitable method as known in the art. Methods may include, without limitation, microinjection, transformation, and transfection methods. Transformation or transfection may occur under natural or artificial conditions.

The method for the transformation is not particularly limited. Recipient cells can be treated with calcium chloride so as to increase the permeability thereof for DNA, which has been reported for the Escherichia coli K-12 strain (Mandel, M. and Higa, A., J. Mol. Biol., 1970, 53, 159-162). Competent cells can be prepared from cells, which are in the growth phase, followed by transformation with DNA, which has been reported for Bacillus subtilis (Duncan, C. H., Wilson, G. A. and Young, F. E., Gene, 1977, 1:153-167). Alternatively, DNA-recipient cells can be made into protoplasts or spheroplasts, which can easily take up recombinant DNA, followed by introducing a recombinant DNA into the DNA-recipient cells, which is known to be applicable to Bacillus subtilis, actinomycetes, and yeasts (Chang, S. and Choen, S. N., 1979, Mol. Gen. Genet., 168:111-115; Bibb, M. J., Ward, J. M. and Hopwood, O. A., 1978, Nature, 274:398-400; Hinnen, A., Hicks, J. B. and Fink, G. R., 1978, Proc. Natl. Acad. Sci. USA, 75:1929-1933).

Still further provided is an isolated or purified polypeptide having the amino acid sequence of SEQ ID NO: 4, 5, or 6. “Functional fragments” of the polypeptides are also provided. Desirably, the functional fragment retains at least at least 20%, 40%, 60%, 80%, or 100% of the activity of the full-length polypeptide. Exemplary functional fragments of the polypeptides include highly conserved amino acid residues.

The polypeptides, and functional fragments thereof, may be further modified in vitro or in vivo to include non-amino acid moieties. These modifications may include, but are not limited to, acylation (e.g., O-acylation (esters), N-acylation (amides), S-acylation (thioesters)), acetylation (e.g., the addition of an acetyl group, either at the N-terminus of the protein or at lysine residues), formylation, lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine) are enzymatic or covalent attachments.

Even still further provided is a method of producing (including producing at an increased level compared to a wild-type host cell) phenylalanine in a prokaryotic host. The method comprises culturing an above-described host cell in medium (such as BG-11 medium, in particular BG-11 medium, which has been modified to contain increased concentrations of magnesium sulfate heptahydrate, sodium nitrate, potassium phosphate dibasic, and A5 mineral solution and ammonium chloride for a bacterial host, such as Cyanobacterium) under culture conditions that support the production of phenylalanine. The method can further comprise isolating the phenylalanine, such as by collecting phenylalanine from a biomass of the cultured variant or a culture medium thereof. In embodiments, the prokaryotic host is Cyanobacterium, and the medium is modified BG-11. In embodiments, the BG-11 medium is supplemented with concentrated modified BG-11 medium about every three days. Preferred methods are disclosed in USPAPN 2023/0013336, which was published Jan. 19, 2023, and which is hereby incorporated by reference for its teachings regarding same. Other methods include analytical chemistry techniques, such as high-performance liquid chromatography and other chromatographic techniques.

When two or more of genes are introduced, it is sufficient that each of the genes is expressibly harbored by the prokaryotic host, such as Cyanobacterium. For example, all the genes may be carried by a single expression vector or a chromosome. Further, the genes may be separately carried by two or more expression vectors, or separately carried by a single or two or more expression vectors and a chromosome. An operon comprising two or more genes may also be introduced.

The expression of a gene can be increased by improving the transcription efficiency of the gene. The transcription efficiency of a gene can be improved by, for example, replacing the promoter of the gene on a chromosome with a stronger promoter. The “stronger promoter” means a promoter providing an improved transcription of a gene compared with an inherently existing wild-type promoter of the gene.

The expression of a gene can also be increased by improving the translation efficiency of the gene. The translation efficiency of a gene can be improved by, for example, replacing the Shine-Dalgarno (SD) sequence (also referred to as ribosome binding site (RBS)) for the gene on a chromosome with a stronger SD sequence. The “stronger SD sequence” means a SD sequence that provides an improved translation of mRNA compared with the inherently existing wild-type SD sequence of the gene. Further, it is known that substitution, insertion, or deletion of several nucleotides in a spacer region between RBS and the start codon, especially in a sequence immediately upstream of the start codon (5′-UTR), significantly affects the stability and translation efficiency of mRNA, and hence, the translation efficiency of a gene can also be improved by modifying them.

The translation efficiency of a gene can also be improved by, for example, modifying codons. For example, in the case of heterogenous expression of a gene or the like, the translation efficiency of the gene can be improved by replacing a rare codon present in the gene with a synonymous codon more frequently used. Codons can be replaced by, for example, the site-specific mutation method for introducing an objective mutation into an objective site of DNA. Alternatively, a gene fragment in which objective codons are replaced may be totally synthesized. Frequencies of codons in various organisms are disclosed in the “Codon Usage Database” (http://www.kazusa.or.jp/codon; Nakamura, Y et al, Nucl. Acids Res., 28, 292 (2000)).

Further, the expression of a gene can also be increased by amplifying a regulator that increases the expression of the gene, or deleting or attenuating a regulator that reduces the expression of the gene.

Such methods for increasing the gene expression as mentioned above may be used independently or in an any combination.

An increase in the activity of a protein can be confirmed by measuring the activity of the protein. The 2,4-dienoyl-CoA reductase activity can be measured as, for example, the activity for decomposing 2,4-dienoyl-CoA. The activity for decomposing 2,4-dienoyl-CoA can be measured by a known method, for example, by monitoring a decrease of NADPH accompanying decomposition of 2,4-dienoyl-CoA (Xue-Ying H. E. et al., Eur. J. Biochem., 248, 516-520 (1997)).

An increase in the activity of a protein can also be confirmed by confirming an increase in the expression of a gene coding for the protein. An increase in the expression of a gene can be confirmed by measuring an increase in the transcription amount of the gene, or by measuring an increase in the amount of a protein expressed from the gene.

An increase in the transcription amount of a gene can be confirmed by comparing the amount of mRNA transcribed from the gene with that observed in a non-modified strain such as a wild-type strain or parent strain. Examples of the method for evaluating the amount of mRNA include Northern hybridization, RT-PCR, and so forth (Sambrook, J., et al., Molecular Cloning A Laboratory Manual/Third Edition, Cold Spring Harbor Laboratory Press, Cold Spring Harbor (USA), 2001). The amount of mRNA may increase, for example, 1.5 times or more, 2 times or more, or 3 times or more, as compared with that of a non-modified strain.

An increase in the amount of a protein can be confirmed by Western blotting using antibodies (Molecular Cloning, Cold Spring Harbor Laboratory Press, Cold Spring Harbor (USA), 2001). The amount of the protein may increase, for example, 1.5 times or more, 2 times or more, or 3 times or more, as compared with that of a non-modified strain.

The aforementioned methods for increasing the activity of a protein can be applied to, in addition to the enhancement of the activity of 2,4-dienoyl-CoA reductase, enhancement of the activity of an arbitrary protein such as L-amino acid biosynthesis enzymes and transporters, and enhancement of the expression of an arbitrary gene such as genes coding for the those arbitrary proteins, fad regulon, cyoABCDE operon, PS gene, and PNO gene.

### EXPERIMENTAL

The following serves to illustrate the present disclosure. The examples are not intended to limit the scope of the claimed invention in any way.

### Materials and Methods

### Strains and Standard Culture Conditions

The wild-type strain used was Synechococcus elongatus sp. PCC 11801²⁶ (hereafter PCC 11801), procured from Prof. Wangikar, Indian Institute of Technology, Bombay. The wild type (WT) and all strains developed were routinely cultured in a modified BG-11 medium (hereafter BG-11M) in 250 mL Erlenmeyer flasks with a culture volume of 50 mL at constant temperature 38° C., 240 μmol photons m⁻² s⁻¹ light, 200 rpm with supply of air or 3% CO₂-enriched air in an incubator shaker (Infors HT Minitron) or a room maintained at 38° C. with ˜1,000 μmol photons m⁻² s⁻¹ light, shaken at 200 rpm under ambient CO₂. BG-11M medium was prepared by supplementing BG-11²⁷ with an additional 1 g sodium nitrate L⁻¹, 20 mg magnesium sulfate heptahydrate L⁻¹, 40 mg potassium phosphate L⁻¹, 10 mg ammonium chloride L⁻¹ and 1 ml of A5 trace metal solution L⁻¹.

### Random Mutagenesis, Selection, and Phe Quantification

Random mutagenesis was performed by modifying a previously described protocol²⁸ with methylmethanesulfonate (MMS) and ultra-violet (UV) irradiation as the mutagens. Cells were prepared for random mutagenesis by pelleting exponentially growing cultures (OD ˜0.6-1) of PCC 11801 and resuspending in 1 mL of fresh BG-11M medium. For MMS mutagenesis, 1% (v/v) MMS (Sigma-Aldrich, St. Louis, MO) was added, and the reaction was quenched by the addition of 5% (w/w) sodium thiosulfate solution to yield a ˜85-95% kill rate. For UV mutagenesis, the culture was subjected to different times of UV irradiation (302 nm peak, UVP Transilluminator) to attain a kill rate of ˜90%.

The mutagenized cultures were incubated at 38° C. overnight in the dark before selection. To select for Phe overproducers, concentrations ranging from 0.5-2 mg/ml Phe analog 3-(2-thienyl)-dl-alanine (Sigma-Aldrich, St. Louis, MO) on BG-11M agar plates were applied. The parent strain and mutagenized cultures were diluted and spread on BG-11M (to test kill rate) or BG-11M Phe analog-containing plates and incubated at 38° C. until colonies appeared. Colonies that successfully grew on analog selection plates were picked and re-streaked on selection plates for at least three rounds to enable complete segregation of mutations. After transfer to liquid cultures, Phe was quantified as previously described using LC-MS/MS²⁸. Briefly, 1 mL of culture was centrifuged to remove the supernatant, diluted to an appropriate volume, transferred to a glass vial, and stored at −20° C. for up to one week before injection into the LC-MS/MS instrument.

Phe-overproducing PCC 11801 strains were engineered using random mutagenesis by employing MMS and UV irradiation as mutagens followed by selection on Phe analog 3-(2-thienyl)-dl-alanine. Mutants resistant to analogs can overcome potential feedback regulation leading to overproduction of Phe⁴². Phe analogs can bind to feedback inhibition sites in the shikimate pathway enzymes, inhibiting biosynthesis of aromatic amino acids. Thus, mutants that are resistant to Phe analogs are likely to be insensitive to feedback inhibition by Phe. Mutants (21) resistant to 1, 1.5, and 2 mg mL⁻¹ 3-(2-thienyl)-dl-alanine were screened for Phe production. Almost all the Phe (>95%) was excreted to the media, as previously reported for aromatic amino acid overproducers^{16,28,43}. The three highest Phe-producing mutant strains from both MMS (M1, M4, and M9) and UV mutants (M14, M17, and M21) were further screened in triplicate for Phe production and growth to ensure stable phenotype after removal of selection pressure. In general, mutants created by UV mutagenesis had higher levels of Phe accumulation compared to mutants created by MMS. M14 proved to be the best strain and accumulated 200±40 mg PheL⁻¹ in the supernatant after five days in culture at 240 μmol m⁻²s⁻¹ white light under ambient CO₂.

Multiple rounds of mutagenesis, especially with different mutagens can be crucial to improving the phenotype under selection since different mutagens can explore independent mutation spaces. To improve Phe production, M14 was selected for further mutagenesis as it was the best strain from the first round of random mutagenesis. Random mutagenesis using UV irradiation or 1% MMS (v/v) exposure was tested independently to achieve ˜90-95% kill rate coupled with selection on plates containing 1.5, 2, 2.5, and 3 mg ml⁻¹ of the Phe analog, 3-(2-thienyl)-dl-alanine. After screening several resistant colonies as described previously, only one colony was found to outperform the parent strain M14 for Phe productivity. This colony was a result of UV exposure and selection on 1.5 mg mL⁻¹ analog. This mutant denoted as M14.2, accumulated roughly 2-fold Phe in the media compared to M14.

### Genome Sequencing

Mutant cultures were sent to Purdue Genomics Core Facility for DNA extraction and sequencing using Illumina (NovaSeq 6000) and minION (Oxford Nanopore Technology). De novo assembly of each sequence was conducted using Unicycler²⁹ and single nucleotide polymorphisms (SNPs) and insertions/deletions (INDELS) were called using Genome Analysis Toolkit (GATK) HaploTypeCaller³⁰ using PCC 11801 as the reference genome. NCBI BLASTX³¹ was used to identify mutated protein coding regions in nucleotide sequences centered on each mutation and 101 nucleotides in length.

To identify the genetic underpinning of the Phe overproduction, single nucleotide polymorphism (SNP) and insertion/deletion (INDEL) were analyzed in the mutants. De novo assembly of re-sequenced PCC 11801 as well as M14 and M14.2 showed 100% coverage compared to the published reference genome (ASM384644v1). Comparisons between all three strains identified 59 fully segregated mutations. Mutations found in both M14 and M14.2 are likely responsible for Phe overproduction and mutations found in only M14.2 may be responsible for the subsequent increase. The mutations in the protein coding regions that were identified for the fully segregated mutations in M14 and M14.2 are shown in Table 1.

TABLE 1
Fully segregated SNPs and INDELs in Phe overproducers M14 and M14.2

| Gene | | | | |
|---|---|---|---|---|
| annotation | Protein | M14 | M14.2 | Accession Number |
| PheA | Prephenate dehydratase | G221W | G221W | WP_228383071.1 |
| TolC | TolC family protein | L531W | L531W | WP_261789828.1 |
| KaiA | circadian clock protein | | Y185Lfs | WP_208673134.1 |

Both M14 and M14.2 had mutations in prephenate dehydratase (PD), which is responsible for the conversion of prephenate to phenylpyruvate, which is finally converted into Phe. The SNP within this enzyme lies at position 221, which is in the ACT domain (residue 181-246, Pfam database) that is involved in feedback regulation by amino acids⁴⁴. This mutation may prevent Phe binding to PD and thus removes feedback inhibition of PD, allowing the strains to overproduce Phe. Previous work using random mutagenesis to overproduce Phe in Synechococcus sp. strain PCC 7002⁴⁵ also identified mutations in PD, which further supports that this mutation is responsible for the Phe overproduction phenotype.

In addition, both M14 and M14.2 had mutations in TolC. This protein has been identified as an exporter of toxins, including proteins and antibiotics, due to its role as an integral part of the efflux pump⁴⁶. Mutation in TolC may enhance transport of Phe out of the cell and thereby reduce potential toxicity to the cells due to intracellular Phe accumulation.

Interestingly, the mutation found only in M14.2 included kaiA, which is a well-known protein involved in maintaining a circadian rhythm⁴⁷. However, it is unclear how this mutation directly contributes to the increased production of Phe in M14.2.

### Growth, Dry Cell Weight (DCW) Measurements

The growth of cultures was monitored by optical density at 730 nm (OD₇₃₀) using a Beckman DU Series 500 spectrophotometer. A linear relationship between the dry cell weight (DCW) and optical density was established at 0.40 g (dry cell weight) L⁻¹ OD₇₃₀⁻¹. Briefly, cultures at different stages of growth were pelleted, washed with ultrapure water, and filtered onto pre-weighed 0.8 μm filters and dried at 65° C. until weight remained constant. A linear fit was used to determine the relationship between DCW and OD₇₃₀.

### Carbon and Nitrogen Content of Biomass

Cultures were grown in triplicate, and biomass was dried at 65° C. in clean glass vials until weight remained constant. Samples (0.5 mg) were accurately weighed into tin capsules, and the carbon and nitrogen fractions were analyzed using a Sercon 20-22 elemental analyzer isotope ratio mass spectrometer (EA-IRMS) by the Purdue Stable Isotope Facility. NIST 1547 was used as the standard reference material.

### Chlorophyll a, and Carotenoid Content

Chlorophyll a content and carotenoid content were measured as previously described^{32,33} Briefly, cells were grown at different light intensities (40, 240, and 1000 μmol-photons m⁻² s⁻¹) and harvested at 0.8-1 OD₇₃₀. Culture (2 mL) was centrifuged at 4° C. at 7,000 rpm for 5 mins. Supernatant was removed, and the cultures were resuspended in either chilled methanol or 80% acetone at 4° C. and incubated on ice for 10-20 mins. Samples were centrifuged again, and the absorbance was measured using either a Beckman DU Series 500 or a U-3900 Head-on PMT spectrophotometer. Methanol-extracted chlorophyll a and carotenoids were quantified using equations 1 and 3, respectively, and 80% acetone-extracted chlorophyll a was quantified using equation 2.

$$Chla ( \frac{µg}{ml} ) = 12.9447 ( {A}_{665} - {A}_{720} ) \qquad ( 1 )$$

$$Chla ( \frac{µg}{ml} ) = 12.25 ( {A}_{663.6} - {A}_{750} ) - 2.55 ( {A}_{646.6} - {A}_{750} ) \qquad ( 2 )$$

$$Carotenoids ( \frac{µg}{ml} ) = [ 1000 ( {A}_{470} - {A}_{720} ) - 2.86 ( Chla ( \frac{µg}{ml} ) ] / 221 \qquad ( 3 )$$

The whole cell absorption spectra were obtained for the wild type and M14.2 by recording absorbance at wavelengths between 350 to 750 nm using Molecular Devices SpectraMax M2 spectrophotometer. Each spectrum was normalized to the corresponding optical density at OD₇₃₀.

### Pulse Amplitude Modulated (PAM) Fluorometry

Cultures were grown to an OD₇₃₀ of ˜0.8 in triplicates under either 40, 240 or 1,000 μmol photons m⁻² s⁻¹, depending on the actinic light intensity to be tested. All cultures were centrifuged, and the cells were resuspended in fresh BG-11M media prior to measurements. All PAM measurements were performed in a warm room maintained at 38° C. using a Hansatech pulse amplitude modulated (PAM) fluorescence monitoring system (FMS1) (Norfolk, England) equipped with an emitter-detector unit. A DW2/2 liquid phase electrode chamber (Hansatech Instruments) was used to hold 2 mL of the sample. Different intensities of actinic light (40, 240 or 1000 μmol-photons m⁻² s⁻¹) were provided by internal optics of FMS1. The samples were stirred with an integral magnetic stirrer. Prior to the measurement, all samples were dark adapted for 3 mins of which the last 30 sec was recorded as the minimum fluorescence Fₒ with a measuring light (≤0.01 μmol photons m⁻² s⁻¹). Maximum fluorescence for dark adapted state (Fₘ) was determined using a 0.8 sec saturating pulse of white light (13,000 μmol photons m⁻² s⁻¹). An actinic light of 40 (LL), 240 (ML) or 1000 (HL) μmol photons m⁻² s⁻¹ intensity, depending on the experiment, was then applied. Six additional saturating pulses were given 60 sec apart, and the last pulse was used for determination of Fₘ′ when a steady state fluorescence signal (Fₛ) had been reached. Actinic light was subsequently switched off to determine Fₒ′. Finally, Fₘ^{DCMU} was determined by addition of 20 μM 3-(3,4-dichlorophenyl)-1,1-dimethylurea (DCMU) with an actinic light of 1000 μmol photons m⁻² s^{-1 34}.

Effective quantum yield Φ_{PSII}=(F^{m}′−Fₛ)/Fₘ′, maximum quantum yield (Φ_{PSIImax}=(Fₘ^{DCMU}−Fₒ)/Fₘ^{DCMU}, photochemical quenching q_{P}=(Fₘ′−Fₛ)/(Fₘ′−Fₒ′), estimate of fraction of open PSII centres q_{L}=(Fₘ′−Fₛ)/[(Fₘ′−Fₒ)×(Fₒ′/Fₛ)], non-photochemical quenching NPQ=(Fₘ^{DCMU}−Fₘ′)/Fₘ′, and relative electron transport rate through PSII rETR2=0.5×Φ_{PSII}×PAR were calculated as previously described³⁵⁻³⁷.

### P700 Difference Absorption Spectroscopy

Synechococcus cultures were grown in biological triplicates at 240 μmol photons m⁻² s⁻¹ white light until they reached mid-exponential growth phase. Chlorophyll concentrations were determined as described in the materials and methods above. Samples were then centrifuged and resuspended in fresh BG-11M medium to a chlorophyll concentration of 10 μg/mL. P700 absorbance measurements were made by a JTS-10 spectrophotometer (SpectroLogiX) using a 630 nm actinic light and a 705 nm measuring light. Samples were dark-adapted for 3 minutes and mixed by pipetting right before measurements were taken. Basal P700 absorbance after dark adaptation was monitored with the measuring light on for 10 sec. Samples were subsequently treated for 10 sec with a 720 nm far red light (200 μmol photons m⁻² s⁻¹) to avoid acceptor-side limitation of PSI. Enabling the P700 absorption measurement, this background illumination also induces a donor-side limitation due to its preferential excitation of PSI over PSII. Interestingly, this far-red light source, with a wavelength that overlaps slightly with the measuring light, causes a positive P700 absorption signal by straying into the detector unit. A 630 nm, 250 ms-long saturating pulse (2600 μmol photons m⁻² s⁻¹) was then applied at the end of the far-red light to determine Pₘ, the maximally oxidized P700 signal. Actinic light (45, 320, or 940 μmol photons m⁻² s⁻¹) was immediately switched on for 5 sec to record the intermediately oxidized P700 signal denoted as P. A second saturating pulse was then applied at the end of the actinic light to determine Pₘ′, and the dark re-reduction of P700 was followed for 7 sec. The stable P700 absorbance signal was noted as P₀. Interestingly, Pₘ values were consistently lower than Pₘ′ values, an observation also noted in other work³⁸. Therefore, Pₘ in the presence of 40 μM DCMU and 2 mM hydroxylamine (HA) was estimated as previously described³⁸. The P700 absorption signal was further corrected by the subtraction of a contaminating signal from chlorophyll fluorescence, as determined by the measurement of the sample under actinic illumination without the probe light. Photosystem I quantum yield Y(I)=(Pₘ′−P)/(Pₘ−P₀), quantum yield of non-photochemical energy dissipation due to donor side limitation Y(ND)=(P−P₀)/(Pₘ−P₀), and quantum yield of non-photochemical energy dissipation due to acceptor side limitation Y(NA)=(Pₘ−Pₘ′)/(Pₘ−P₀) were calculated as previously described³⁹. The sum of Y(I), Y(ND), and Y(NA) is one. Relative electron transport rate through PSI was calculated as rETR1=0.5×Φ_{PSI}×PAR.

The percentage of cyclic electron flow and total electron flow were determined as described previously from P700 re-reduction kinetics by fitting a first order exponential decay equation to determine half times⁴⁰. P700 oxidation kinetics were also determined similarly by fitting a first order reaction model to determine the half times as previously described⁴¹. To measure cyclic electron flow around PSI, linear electron transport was blocked by the addition of PSII-specific inhibitors 40 μM DCMU and 2 mM HA. Measurements were also carried out under 40 μM dibromothymoquinone (DBMIB), a cyt b₆f complex inhibitor that eliminates both linear and cyclic electron transport pathways.

### Characterization of Phe-Overproducing Mutants Under Different Conditions

The performance of mutant strains was tested under 12h:12h diurnal cycle, akin to what might be expected in outdoor culture conditions. Both M14 and M14.2 were adapted to at least one subculture under diurnal cycle before being tested for Phe production and biomass accumulation. The Phe productivity normalized to time under illumination for M14 and M14.2 in a 12h:12h diurnal cycle was comparable to continuous illumination. The biomass accumulation for both mutant strains was comparable to wild type. This shows the promise of the strains under outdoor relevant diel conditions, where the Phe production phenotype is maintained. The disruption of kaiA in M14.2 had no apparent growth phenotype.

Enhancing the dissolved concentration of CO₂ has been shown to increase not only the growth rate but also the final culture density of PCC 11801³⁰. Previously, aromatic amino acid production was found to be enhanced under the supply of CO₂ enriched air^{28,43}. To improve final product and biomass titer and productivities, the effect of a 3% CO₂ supply was tested.

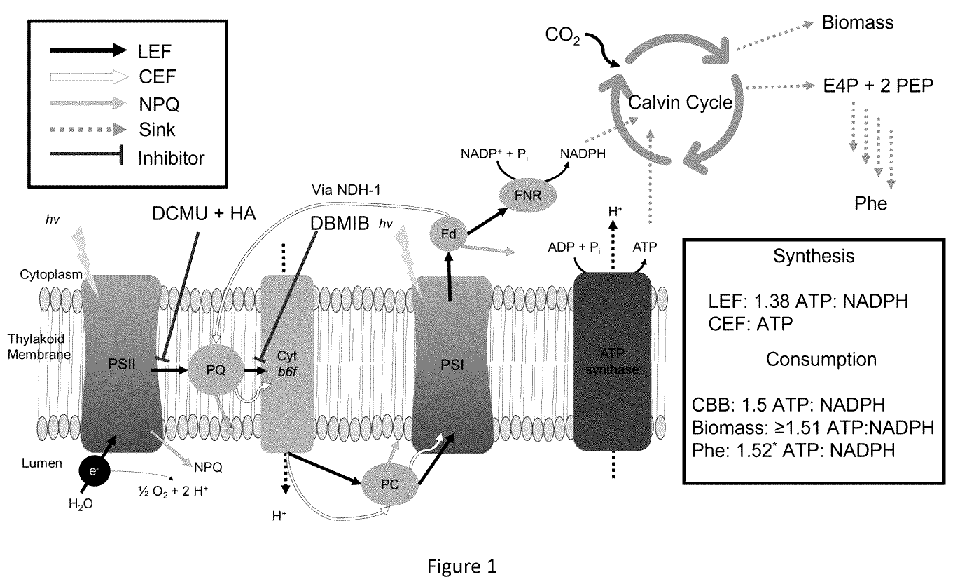

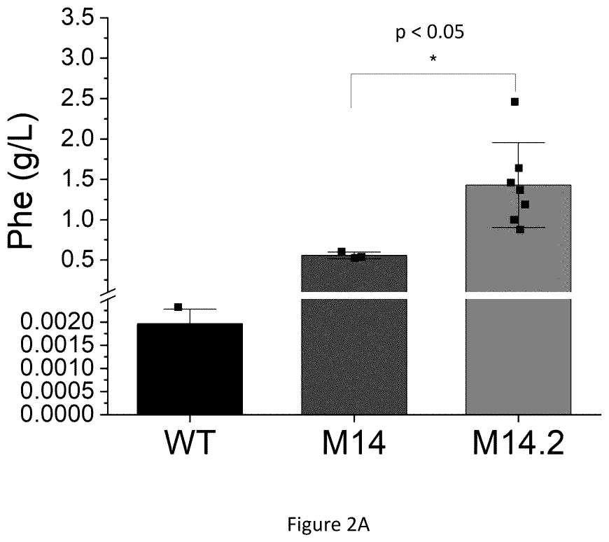

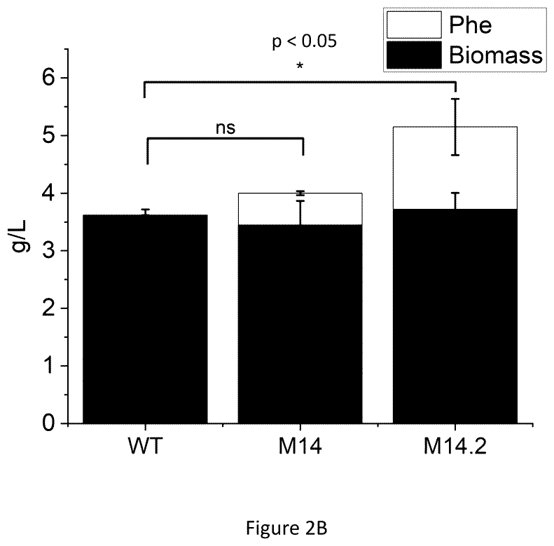

FIG. 2A shows the Phe titers of mutant strains. To ensure stability of the most productive strain M14.2, the experiment was repeated three times, and it was seen that M14.2 had a consistent phenotype with a titer 1.24±0.13 g Phe l⁻¹ in three days. FIG. 2B shows the total accumulation of two major sink products, biomass and Phe.

Improving biomass accumulation is vital to increase the titer of growth associated products such as Phe. Recently, the biomass accumulation of Synechocystis sp. PCC 6803 and Synechococcus sp. PCC 11901 was improved by alleviating nutrient limitations^{48,49}. Optimization of the contents of BG-11M was tested by further increasing levels of nitrates, phosphates, and sulfates as well as nutrient-replete 5×BG-11M. Interestingly, none of these resulted in improved performance compared to BG-11M media. This may be due to multiple nutrient limitations that cannot be removed by optimization of nutrients one at a time or, in the case of 5×BG-11M, toxicity due to high cation concentration. The addition of complete BG-11M nutrients every three days was tested using a concentrated solution to minimize media volume changes. This strategy enabled increasing the biomass and Phe accumulation to nearly 7 g DCW l⁻¹ and 3 g Phe 1-1 respectively in 15 days under 240 μmol-photons m⁻² s⁻¹ light and 3% CO₂. This result also outlines a strategy that can be potentially implemented at higher scales to improve final Phe titer.

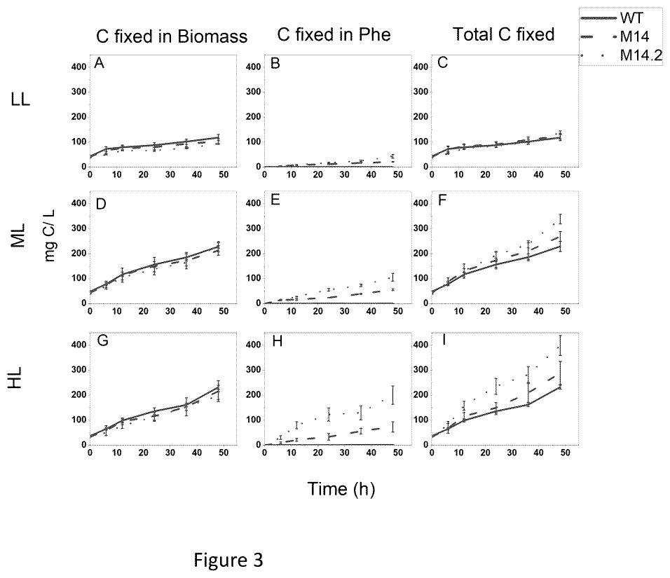

Both M14 and M14.2 showed higher total carbon productivities (FIG. 3) compared to wild type, and there was no significant difference in biomass accumulation. These three strains (WT, M14, M14.2) were used to investigate source-sink dynamics.

FIG. 3 shows the contribution of Phe and biomass sinks to the total carbon fixed under different light conditions in WT and Phe sink-engineered strains M14 and M14.2. The total carbon fixed was calculated by determining the carbon content in the two sinks, biomass, and excreted Phe (C %=65.43). To determine the carbon content in biomass of different strains, elemental analysis of biomass was performed and the estimated C % and N % is shown in Table 2. Data represent the mean and standard deviation of three biological replicates for M14 and M14.2, and two replicates for WT grown at 38° C. and 240 μmol photons m⁻²s⁻¹ light intensity at ambient CO₂. p-values based on two tailed t-test in comparison to WT are reported in parenthesis. The mean C % and N % for both M14 and M14.2 were lower than that of the WT. However, these differences were not statistically significant, ruling out the possibility that Phe overproduction was achieved by partially diverting carbon and nitrogen from the biomass to the Phe sink.

TABLE 2
Carbon and Nitrogen content of biomass in WT, M14, and M14.2.

| Strain | Carbon (%) | Nitrogen (%) |
|---|---|---|
| WT | 50.60 ± 1.00 | 11.56 ± 0.16 |
| M14 | 47.4 ± 1.06 (p = 0.08) | 11.38 ± 0.21 (p = 0.508) |
| M14.2 | 48.20 ± 0.65 (p = 0.09) | 11.11 ± 0.31 (p = 0.208) |

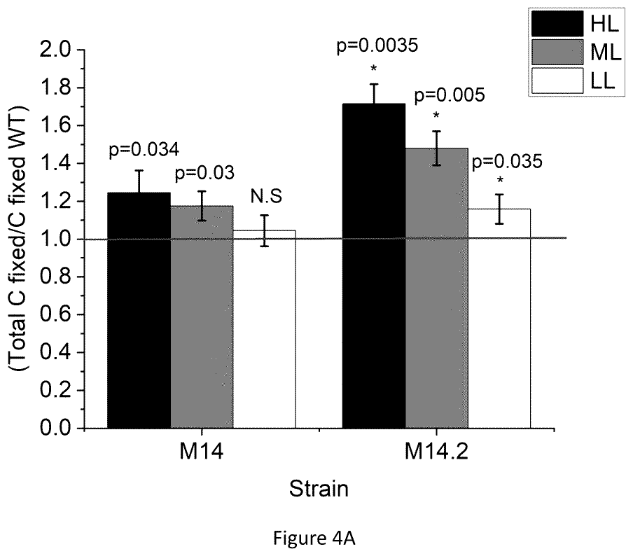

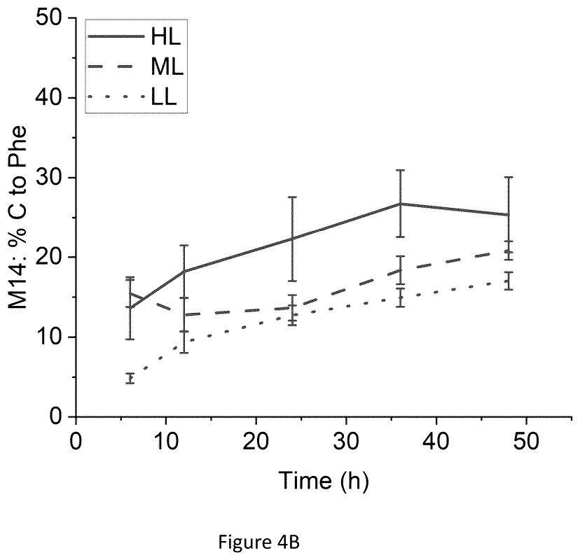

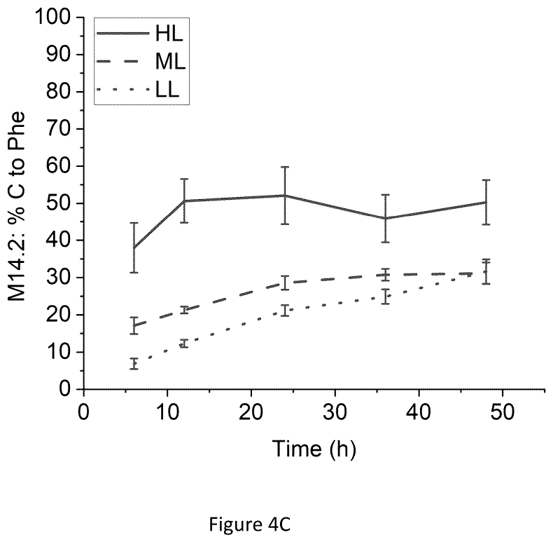

FIG. 3 shows that the total carbon fixation increases with increase in light intensity in all three strains as expected. All three strains show highest carbon fixation in the high light intensity (HL) condition, which was previously reported for PCC 11801²⁶. Under medium light intensity (ML) condition and HL condition, biomass production is not affected with the introduction of Phe sink (FIG. 3, graphs D and G), and the Phe production is achieved entirely by enhancing the carbon fixation (FIG. 3, graphs E and H). Under low light intensity (LL) condition (sink limitation), the Phe sink causes a slight reduction in the biomass accumulation (FIG. 3, graph A), while the total carbon fixed is not affected in M14 under the same light condition (FIG. 4 A). M14.2, interestingly, shows a minor increase in total carbon fixed in LL condition (p=0.035) (FIG. 4 A), likely due to contribution from Phe sink (p=0.006) (FIG. 3, graph B).

The results support the hypothesis that, under light limited conditions, the competition between biomass and Phe sinks for energy and reducing power results in reduced biomass accumulation. The results also show that, under excess light, the enhancement in total carbon fixation is dependent on the strength of the engineered sink. Under ML condition and HL condition, a 17% improvement and a 24% improvement in total carbon fixation in M14 over the WT, respectively, were observed, whereas M14.2 showed a 47% improvement and a 70% improvement. Under excess light no significant improvement in biomass accumulation in the sink engineered strains was observed (FIG. 3, graphs D and G). All the improvement in carbon fixation, instead, was directed to the engineered Phe sinks. The light intensity thus directly affects the carbon partitioning (FIGS. 4B and 4C).

### Phe Sink-Engineered Strains Showed Improvement in PSII Efficiency

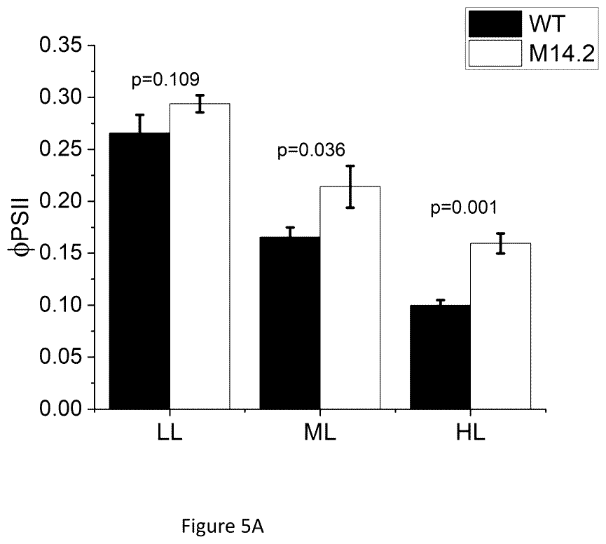

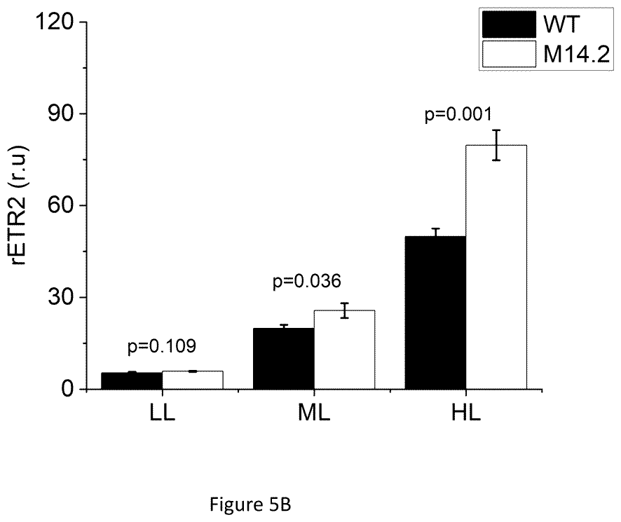

To investigate the effect of the Phe sink on photosynthetic efficiency, the M14.2 strain, which shows a higher flux to Phe compared to M14, was examined. The effective quantum yield of PSII (Φ_{PSII}) and the relative linear electron flow rate through PSII (rETR2) were measured from room temperature chlorophyll fluorescence yield. Cultures were first grown in triplicates under LL, ML, and HL conditions to match the three actinic light intensities used for Chlorophyll fluorescence measurements. With increasing growth (and actinic) light intensity, an expected reduction in the quantum yield of PSII in both WT and Phe overproducer M14.2 was observed (FIG. 5A). However, the mutant showed a higher PSII operating efficiency than WT at all light levels, although significant improvement over WT was seen only at ML and HL (FIG. 5A).

With the introduction of the Phe sink, Φ_{PSII} and rETR2, a measure of the linear electron flow, improved by ˜30% under ML, whereas a 59% improvement was observed under HL. This is comparable to the 47% and 70% enhancement observed in the carbon fixation under the same light conditions at the end of two days of growth. Since these measurements were performed on cultures with the same optical density, it is possible that M14.2 has a higher chlorophyll a content and, therefore, an improved light utilization. The chlorophyll a content of the WT and M14.2 is, however, not significantly different under any of the light conditions, ruling out this possibility.

One pathway through which the quantum yield of PSII could be improved is by decreasing the thermal dissipation of absorbed light energy as nonphotochemical quenching. The introduction of the Phe sink in M14.2 might reduce nonphotochemical quenching especially at high light conditions. However, no significant difference in NPQ between WT and M14.2 at any light levels was found (Table 3). The qP and qL parameters, which reflect the fraction of oxidized Q_{A} electron acceptor of PSII, does not differ between WT and M14.2 at LL and ML but are 42% and 46% higher under high light condition in M14.2. In general, both qP and qL decrease (Q_{A} becomes more reduced) with increase in light intensity, in turn decreasing the openness of PSII reaction centers. At high light, M14.2 has a significantly higher qP or qL compared to WT, as indicative of its higher fraction of open PSII reaction centers available for photochemistry. Engineering of the Phe sink thus results in a greater PSII photochemical efficiency in M14.2 (Table 3).

TABLE 3
PSII photochemical parameters of WT and M14.2.
Each measurement represents the mean and standard deviation of three biological replicates.

| | WT | | | M14.2 | | |
|---|---|---|---|---|---|---|
| Strain | LL | ML | HL | LL | ML | HL |
| NPQ | 0.07 ± 0.02 | 0.08 ± 0.02 | 0.12 ± 0.01 | 0.05 ± 0.02 | 0.15 ± 0.08 | 0.16 ± 0.03 |
| qP | 0.70 ± 0.02 | 0.62 ± 0.05 | 0.24 ± 0.02 | 0.68 ± 0.06 | 0.63 ± 0.01 | 0.34 ± 0.03 |
| 1-qP | 0.3 ± 0.02 | 0.38 ± 0.05 | 0.76 ± 0.02 | 0.32 ± 0.06 | 0.37 ± 0.01 | 0.66 ± 0.03 |
| qL | 0.83 ± 0.02 | 0.71 ± 0.04 | 0.37 ± 0.02 | 0.85 ± 0.02 | 0.75 ± 0.02 | 0.54 ± 0.03 |
| 1-qL | 0.17 ± 0.02 | 0.29 ± 0.04 | 0.63 ± 0.02 | 0.15 ± 0.02 | 0.25 ± 0.02 | 0.46 ± 0.03 |
| Φ_{PSIImax} | 0.42 ± 0.01 | 0.43 ± 0.02 | 0.49 ± 0.01 | 0.46 ± 0.01 | 0.43 ± 0.02 | 0.54 ± 0.01 |

Carotenoids function as accessory light harvesting pigments⁵⁰ and are also involved in photoprotection by quenching of excess light^{51,52}. Thus, the carotenoid content in WT and a Phe sink-engineered strain grown at different light intensities was measured. Interestingly, total carotenoids increased significantly in M14.2 under all light conditions. Although the effect of increased carotenoid content needs to be studied further, it is possible that this helps with light harvesting and improves photoprotection in the mutant.

### Introduction of Phe Sink Suppresses Cyclic Electron Flow

By generating additional ATP molecules, the cyclic electron flow can aid in balancing the ATP/NADPH production and demand¹⁹. To understand the effect of introduction of Phe sink on linear and cyclic electron flow, and thus ATP/NADPH production, P700 difference absorption spectroscopy was used.

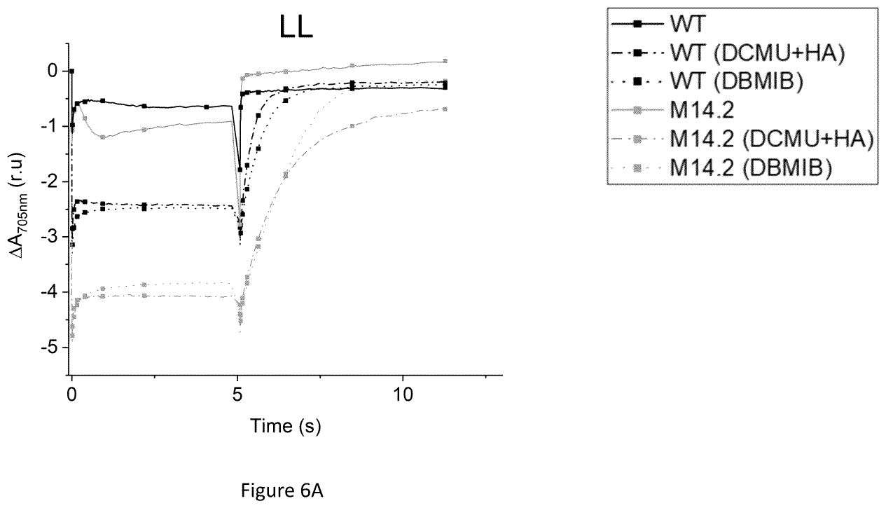

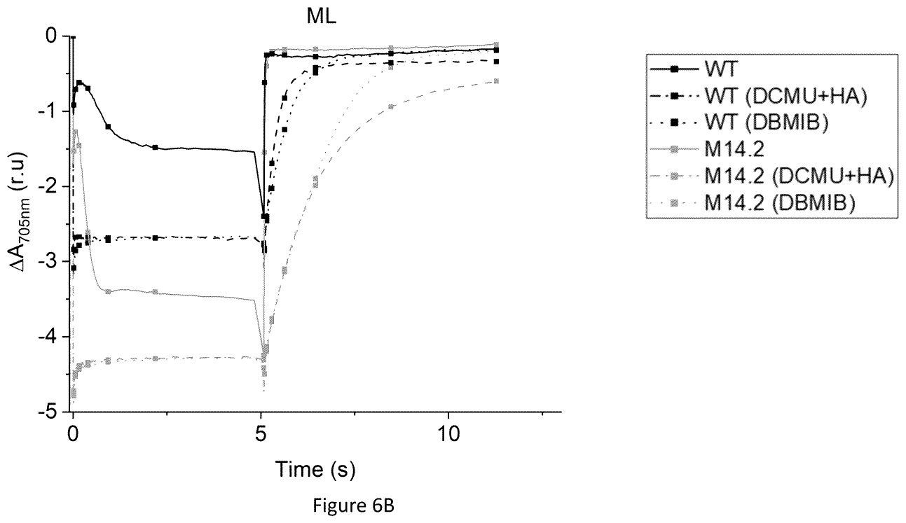

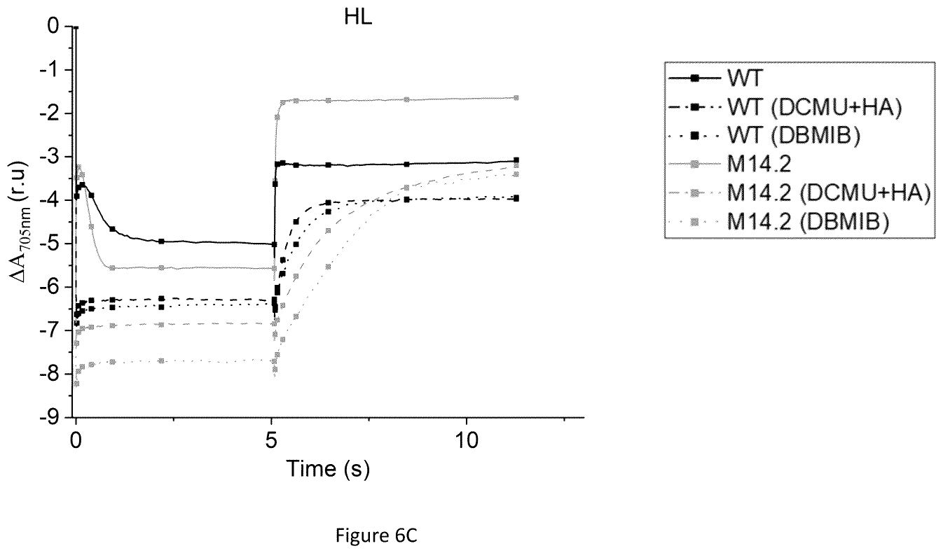

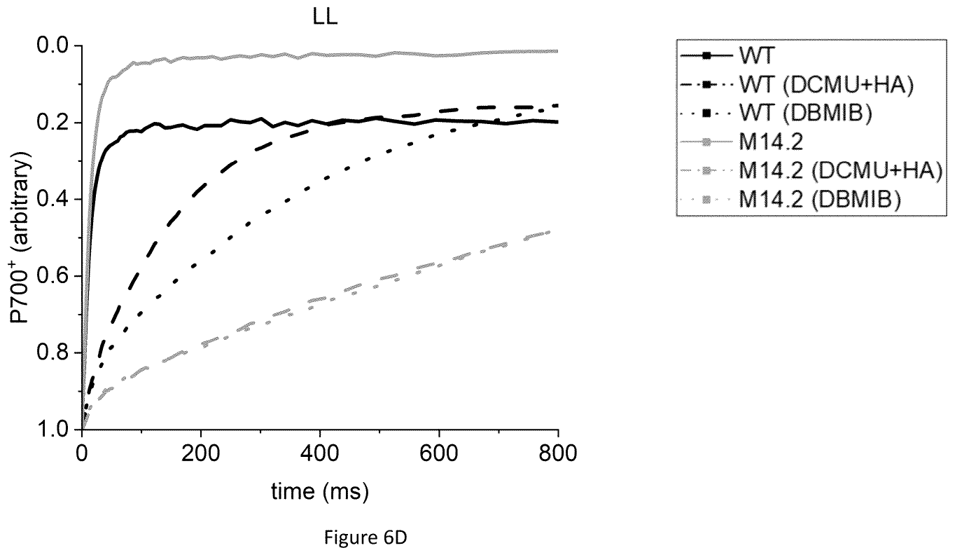

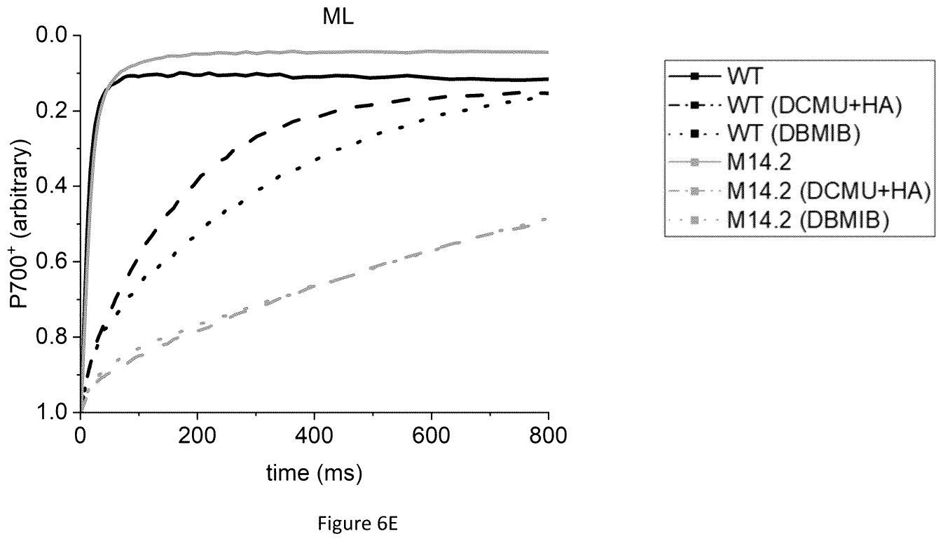

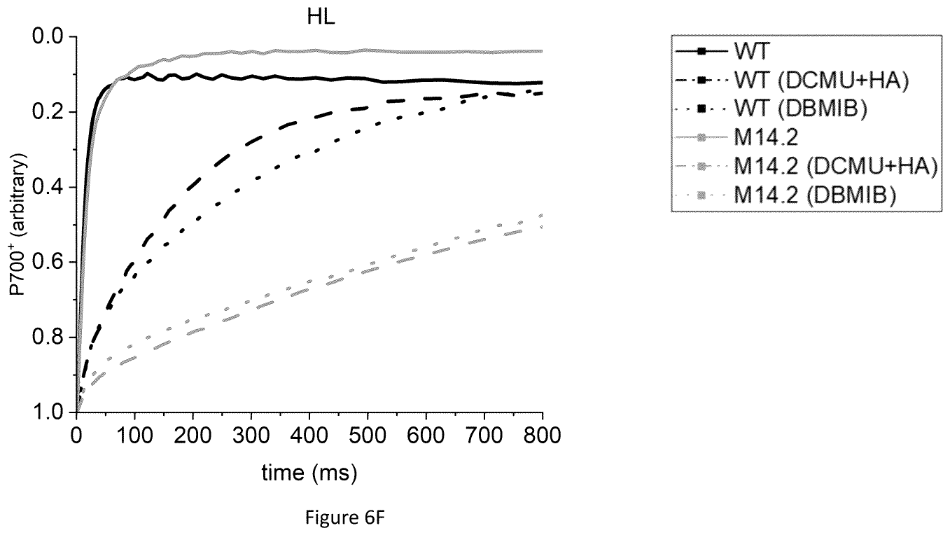

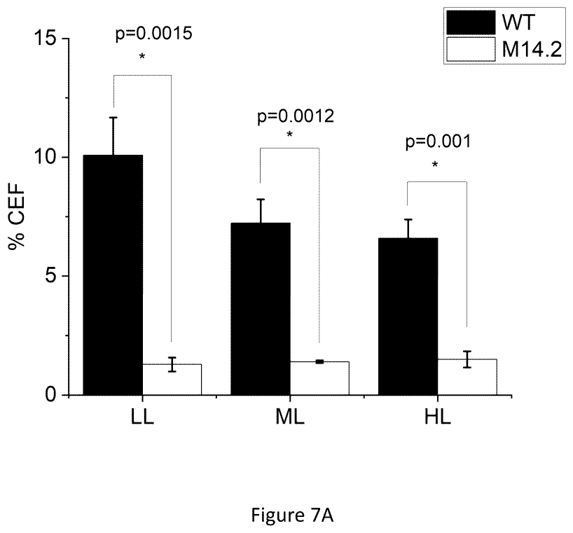

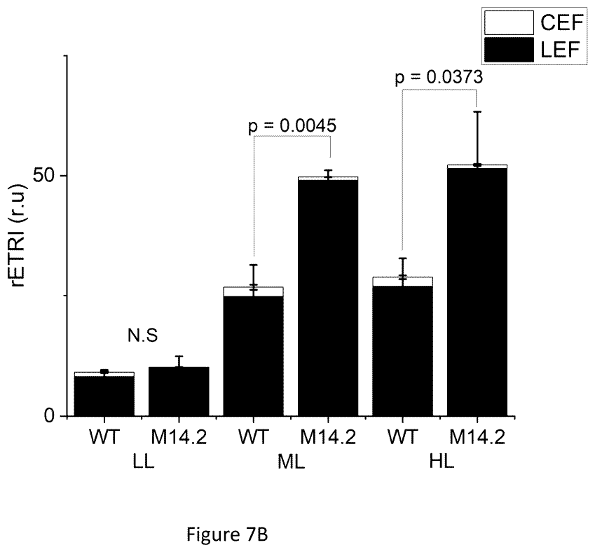

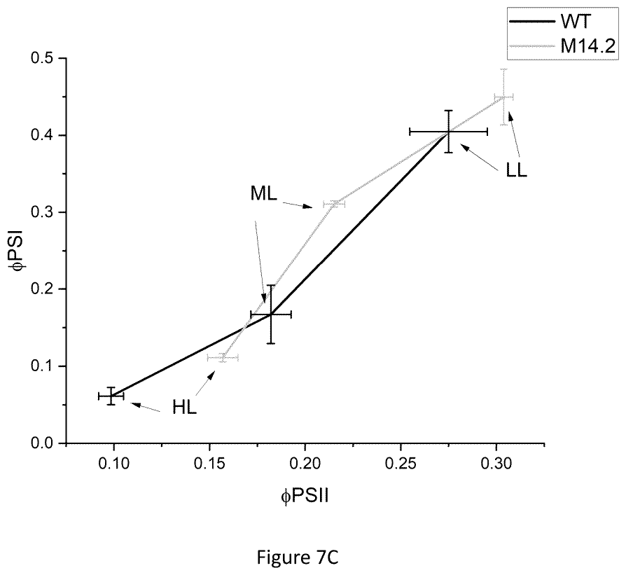

CEF can be estimated by blocking the linear electron flow from PSII using the PSII-specific inhibitors DCMU and HA. The dark re-reduction rate of P700 was measured in the absence (LEF+CEF) and in the presence of DCMU and HA (CEF) to calculate the percentage of CEF (% CEF) in WT and M14.2 under LL, ML and HL conditions (FIG. 7A). The % CEF in WT is higher than M14.2 at all light levels. The re-reduction of P700 is significantly faster in DCMU and HA-treated WT compared to the corresponding M14.2 sample (FIGS. 6D-6F). In the mutant, the % CEF is reduced to nearly 1% or to a negligible rate, similar to the DBMIB-treated sample, due to the introduction of the Phe sink. Since DBMIB treatment should inhibit both linear and cyclic electron flow, the P700 re-reduction rate under this inhibitor likely originates from charge recombination events in P700 or to a minor degree, incomplete inhibition of cyt b₆f. Since CEF generates only ATP, the decrease in % CEF by the introduction of the Phe sink will result in a decrease in the ATP/NADPH production in M14.2. This indicates that, under the growth light intensities tested, Phe sink is likely to be more NADPH-intensive compared to the biomass sink. The results do not indicate any change in % CEF in both WT and mutant as a function of light intensity. In WT, the only major sink is biomass and thus the demand for ATP and NADPH should be the same under different light intensities as the biomass compositions are unchanged. In M14.2, the flux towards the Phe sink is light intensity-dependent (FIG. 4C). Since the ATP and NADPH requirement for biomass and Phe sink are different (FIG. 1), the change in flux to the Phe sink under ML and HL will change the net demand for ATP and NADPH. An increase in % CEF with light intensity might meet any increased demand for ATP. However, no change in % CEF with light intensity was found in M14.2 (Tukey's test for multiple comparison). FIG. 7B shows the relative electron transport through PSI (rETR1) to be significantly higher in the mutant at both ML and HL conditions than WT, with most of the increase in rETR1 arising from the increased LEF. The rETR1 and ΦPSI, the photochemical efficiency of PSI, reflect the rate of both linear and cyclic flow while rETR2 and ΦPSII report only on the LEF. The enhancement in total electron flow, as apparent from rETR1, is 86% at ML and 83% at HL, comparable to the enhancement in carbon fixation (FIG. 3). A plot of the ΦPSI vs ΦPSII also supports the shift towards LEF in M14.2 at HL conditions (FIG. 7C).

### The Impact of Phe Sink on PSI Photochemical Efficiency

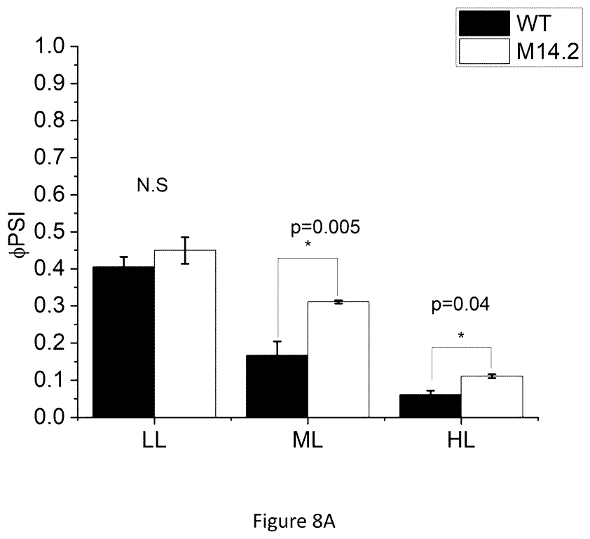

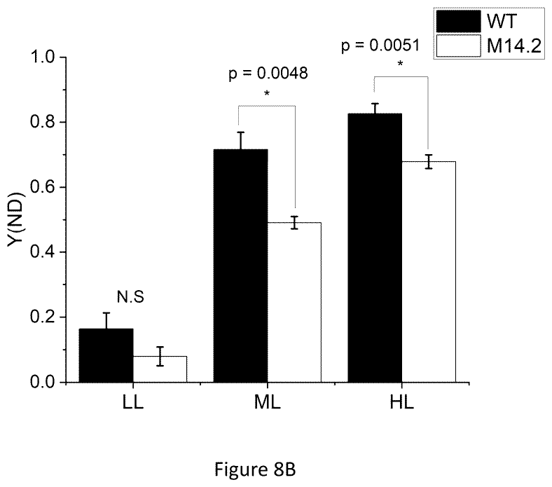

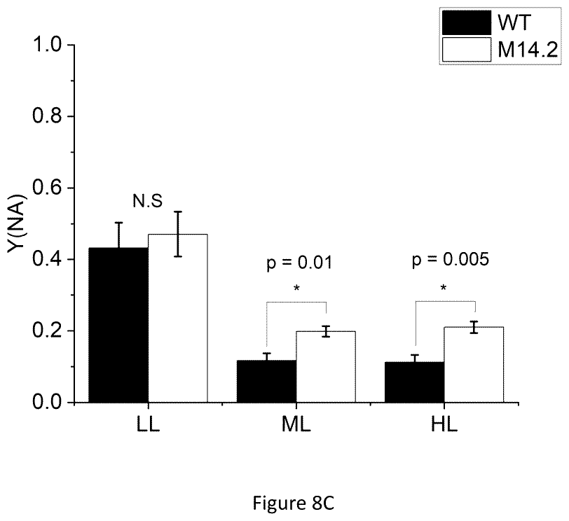

The introduction of Phe sink led to a substantial reduction in % CEF (FIGS. 6A-6F). However, it is unclear how the Phe sink affects the PSI photochemical efficiency (ΦPSI). Therefore, the quantum yield of PSI from P700 redox kinetic data was determined (FIGS. 6A-6F). As expected, ΦPSI of both the WT and M14.2 decreased with increasing light intensity. However, the extent of the ΦPSI decrease at ML and HL was significantly lower in M14.2, compared to WT (FIG. 8A), correlating with the mutant's higher electron flow through PSI at these light intensities (FIG. 7B). A decrease in ΦPSI may arise from an overreduction at the acceptor side of PSI (acceptor side inhibition, Y(NA)) or from a reduced electron flow from the donor side (donor side inhibition or photosynthetic control, Y(ND)). Since the introduction of the Phe sink generates a greater sink demand, it was hypothesized that M14.2 would have a lower PSI acceptor side limitation compared to WT, especially under high light conditions. At low light condition, no difference in acceptor side limitation between WT and M14.2 was found (FIG. 8C). Surprisingly, the introduction of the Phe sink led, instead, to a slight increase in acceptor side limitations at ML and HL conditions. However, the largest loss in PSI efficiency at higher light intensities in both WT and M14.2 was due to donor side limitation. Nevertheless, the donor side limitation was considerably lower in M14.2 compared to WT at ML and HL, thus accounting for its higher (ΦPSI in those light conditions.

### Discussion

Cyanobacteria are increasingly investigated as a sustainable biochemical factory to produce high value products due to their ability to use CO₂ as the sole carbon source¹. Several studies have engineered cyanobacteria for the production of biofuels and biochemicals; however, there are still hurdles to overcome before their industrial application becomes feasible³. One way to improve the feasibility of cyanobacteria for industrial production is to increase their rate of carbon fixation, with the assimilated carbon diverted to desired product. The recent demonstration of improved carbon fixation upon introduction of a heterologous sink in cyanobacteria is an important advance in the field⁵³.

Random mutagenesis on PCC 11801 was used to develop Phe-overproducing strains that can accumulate 3 g Phe L⁻¹, the highest reported titer in cyanobacteria. Our strain M14.2, shows a higher productivity under both ambient and 3% CO₂ conditions with a maximum productivity about 400 mg Phe L⁻¹d⁻¹ under a three-day cultivation cycle. This is significantly higher than the production of Phe reported in Synechocystis 6803, which reached 90.4 mg L⁻¹d^{-1 43} and 152.5 mg L⁻¹d^{-1 16} due to the choice of a fast growing cyanobacteria. The comparison with heterotrophs is not a direct one. Heterotrophic Phe producers, such as E. coli, need to utilize sugars such as glucose and suffer from low yields on glucose, typically lower than 50% of the maximum theoretical yield of 0.55 g Phe per g glucose⁵⁴. Thus, we compare the two-step process of sugar production by crops and subsequent conversion to Phe by heterotrophs with the single step cyanobacterial process using space-time yield as previously described⁵⁵. The current best Phe-producing bacterial strain accumulates roughly 72 g Phe L⁻¹ Phe in medium with a yield of 0.26 mol Phe per mol glucose⁵⁴. Assuming the average yield for sugarcane over the last 10 years (70.63 tfw/ha/annum) and a sugar content of 15%, the space-time yield for the two-step process is 6.91 kg Phe ha⁻¹d⁻¹. In case of cyanobacterial photoautotrophic Phe production, we assume commercial photobioreactors of scale 500,000 L ha^{-1 56}. If we assume the same productivity as observed in diel shake flask experiments of −200 mg Phe produced in three days (66 mg Phe L⁻¹ d⁻¹), it results in a space-time yield of nearly 33 kg Phe ha⁻¹d⁻¹, i.e., 4.8-fold more efficient than the current heterotrophic process in terms of land use.

Future work will target further increasing flux to Phe. Previously, it has been shown that nearly 50% of CO₂ fixed can be directed to Phe⁴³, which indicates that there is further room for improvement. This can be addressed by combining metabolic engineering approaches as previously described for tryptophan production²⁸. It is surprising to see that no SNPs were identified in DAHPS, the first step that drives flux into the shikimate pathway. A similar observation was found in a tryptophan-overproducing mutant in Synechocystis PCC 6803²⁸. In contrast, Synechocystis PCC 6803 mutants that overproduced Phe obtained through adaptive laboratory evolution all had mutations in DAHPS¹⁶. Instead, herein overproduction of Phe was enabled by a SNP in the ACT (feedback regulated domain) of the prephenate dehydratase. A similar deregulation of prephenate dehydratase was observed in C. glutamicum, where mutations occurred in the Arg-202 and Gly-224 residues in the ACT domain⁵⁷. Additionally, M14.2 had a SNP in TolC, a protein previously found crucial to cysteine export in E. coli⁴⁶. TolC forms a channel in the periplasm connecting the outside of the cell with inner membrane bound transporters. TolC-mediated efflux of phenylalanine has not been previously reported. The identified SNP in the TolC protein may improve Phe efflux to the extracellular space, thereby improving Phe production in M14.2.

Previously, it has been suggested that a heterologous sink is necessary to relieve sink limitation⁵⁸. However, even an overflow homologous sink, such as Phe, can result in enhanced carbon fixation. Herein, the role of light intensity in enhancement of carbon fixation is highlighted. The Phe sink competes with biomass under LL condition whereas, under ML condition and HL condition, the enhanced carbon fixation is directed entirely to the engineered sink. This contrasts with studies utilizing 2,3 BD and sucrose sinks, which showed a reduction in biomass accumulation, although the total carbon fixation increased by introduction of the new sink^{11,13,53} Production of Phe-derived 2-phenylethanol also improved total carbon fixation. However, there was a competition between biomass and product sinks in the first four days but, after four days, the enhancement in carbon fixation was directed solely towards the new product sink¹⁴. These observations indicate that the nature of the sink product can influence the behavior of the sink. Products, such as 2-phenylethanol¹⁴ and 2,3 BD¹³, can be toxic to cyanobacteria, while the sucrose sink¹¹ can induce osmotic shock resulting in competition between biomass and heterologous sink, even when light is not limiting. In contrast, an endogenous (homologous) product, such as Phe, does not compete with biomass under light-sufficient condition. Changes in light intensity significantly affected the carbon partitioning to the Phe sink. This is similar to enhancement in carbon assimilation attained in 2,3 BD production in S. elongatus 7942, when light intensity was increased from 50 μmol photons m² s⁻¹ to 250 μmol photons m⁻² s^{-1 13}.

It is hypothesized that the excess absorbed light energy that is otherwise dissipated as non-photochemical quenching can be utilized by the introduction of an additional carbon sink¹¹. The data support this idea as improved quantum yield of PSII in Phe-overproducing strains was found under ML and HL conditions (FIGS. 5A-5B). The photochemical quenching (qP) also improved substantially under high light in the mutant strains. These results are similar to the observations made in sucrose¹¹ and glycerol-producing cyanobacteria strains¹⁵. The improvement in PSII quantum yield leads to increased LEF (FIG. 5B), which, in turn, meets the increased demand for ATP and NADPH created by the Phe sink.

The data reveal that % CEF is greatly reduced in response to the introduction of the Phe sink under all light conditions. This suggests that a lower ATP/NADPH demand might be sufficient for the partitioning of carbon to Phe sink compared to the biomass sink, which is the only carbon sink in WT. Because of unknown and difficult to measure growth-associated and non-growth-associated maintenance costs, it is difficult to estimate accurately the ATP/NADPH demand for biomass accumulation, but it requires a minimum ATP/NADPH of 1.51²⁰. However, previous flux balance models predict a higher demand of 1.7359. The estimate for Phe synthesis is 1.52 but this could be slightly higher if an ATP-dependent amino acid transporter is taken into account⁶⁰. The results indicate that the ATP/NADPH demand for biomass is higher than Phe sink as evident from the lower % CEF in the Phe-overproducing strains. This observation is also consistent with the fact that Phe represents a more reduced carbon molecule (degree of reduction=4.44) than biomass (degree of reduction=4.2), requiring more reductant than ATP (FIG. 1).

The decrease of CEF in the mutant may lead to relaxation of an important safety mechanism of photosynthesis known as photosynthetic control^{40,61}. In photosynthetic control, the increased acidification of the thylakoid lumen at high light intensities slows down the oxidation of PQH₂ at the Qₒ-site of cyt b₆f complex, which, in turn, decreases the inter-photosystem electron transport. CEF is a key contributor towards proton gradient formation and photosynthetic control, which protects PSI from photoinhibition⁶¹. The establishment of photosynthetic control is evident in the increased donor side inhibition of PSI at ML and HL in both WT and M14.2 (FIG. 8B). However, the substantial decrease of CEF leads to lower photosynthetic control and donor side inhibition of PSI in M14.2 (FIG. 8B). This sped up the LEF in M14.2 at ML and HL (FIG. 7B).

The low light data suggest that there is a competition for cellular energy between biomass and Phe production (FIG. 3). This could reduce biomass accumulation and strain robustness under dynamic outdoor growth conditions.

### Statistical Analysis

All data are shown as mean values with errors representing standard deviation of three or more biological replicates unless stated otherwise. Difference between measurements is compared by unpaired student's t-test unless stated otherwise. Statistical analyses were carried out with either Microsoft Excel or by Origin 2021b (OriginLab). The criteria for statistical significance were set as p<0.05.

### Enumerated Embodiments

EE1. An isolated or purified polynucleotide encoding a prephenate dehydratase having an amino acid sequence that is at least about 90% identical to the amino acid sequence of SEQ ID NO: 1:
1 MAALRFQAWL TDQDQQPSHL LACRSIPATL QTLADGTVDY AVVPVENSVE GSVAATLDSL
61 WQLPQLSIQR ALILPIAHAL ISFEDDRTAI RQVLSHPQAL AQCQQWLQRQ LPQAELIPTN
121 STTEALQDLE RHPQRAVIAS TRAAELYQMP IQSFPINDSP DNRTRFWVVS RNLTPGGACT
181 SLSFSLDANV PGALVKPLQI LAERQINLSR IESRPTKRSL [symbol] EYLFFLDLE ADLREPAIAQ
241 AVQAVADCTE QLRVLGSYDS LDFTQVVQPS [SEQ ID NO: 1], in which the glycine at amino acid position 221 (G221) is substituted with an amino acid having an aromatic R group.
EE2. The isolated or purified polynucleotide of EE1, wherein G221 is substituted with an amino acid selected from phenylalanine, tyrosine, and tryptophan.
EE3. The isolated or purified polynucleotide of EE2, wherein G221 is substituted with tryptophan (G221W) [SEQ ID NO: 4].
EE4. An isolated or purified polynucleotide encoding a TolC family protein having an amino acid sequence that is at least about 90% identical to the amino acid sequence of SEQ ID NO: 2:
1 MAAFLYRLSL LSALAIAAHG MEPPAAIADI VDPQATGPSP TIAQNSPPPA ATTPAPTTPP
61 SSPVKEVVPD ANLLKELQAN PNPFQLPNQP NQVQTESLQP LTLEQALNLA RLNNPQVQIR
121 QLQVEQRRAA LRGTEAALYP TLGLQGNAGY QQSGNRLNVT EGSTVQPTGS SLFTTLGQSS
181 IGATLNLNYT IFDFVRGAQL AASRDQVTQA ELDLEATLED LQLTVSEAYY QLQNADQLVR
241 IARESVVASE RSLKDAEALF RAGVGTQFDV LRQQVQLAQD QQNLVDSIGN QDKARRSLVQ
301 ALNLPQNVNV LTADPVELAA PWNLSLDESI VLAFQNRPEL EREVLQRNIS YNQAQAARGQ
361 ILPQLGLQAS YGVTGSINSN LRSGSQALTF PSPTLTNNSN YSYSIGLVLN VPLFDGGLAN
421 ANAQQQELNG QIAEQNFVLT RNQIRTDVET AFYDLQTNLA NIGTTRKAVE QAREALRLAR
481 LRFQAGVGTQ TEVIDSQRDL TRAEANALNA ITAYNLALAR IKRAVSNVSN [symbol] ARVGS [SEQ ID NO: 2], in which the leucine at amino acid position 531 (L531) is substituted with an amino acid having an aromatic R group.
EE5. The isolated or purified polynucleotide of EE4, wherein L531 is substituted with an amino acid selected from phenylalanine, tyrosine, and tryptophan.
EE6. The isolated or purified polynucleotide of EE5, wherein L531 is substituted with tryptophan (L531W) [SEQ ID NO: 5].
EE7. An isolated or purified polynucleotide encoding a circadian clock protein having an amino acid sequence that is at least about 90% identical to the amino acid sequence of SEQ ID NO: 3:
1 MLSQIAICIW VESTAILQDC QQALAGDRYQ LQICSSGDLL LDYAQTHRDQ IDCLLLVATN
61 PGCKTVIQQL CFQGIVVPAI VVGDRDTDDV TDTQKDWVYH SAELHLGIHQ LEQLPYQVDA
121 ALAEFLRQAP VETIADQVML MAATHDPELA SHQRDLAQRL QERLGYLGVY YKRDPDRFLR
181 NLPA [symbol] EGQKL LEAMQTSYRE IVLSYFSPNS NLNQSLDNFV NMAFFADVPV TQVVEIHMEL
241 MDEFAKKLRV EGRSEDILLD YRLTLIDVIA HLCEMYRRSI PRET [SEQ ID NO: 3], in which there is a frameshift mutation at tyrosine at amino acid position 185 (Y185) resulting in the substitution of Y185 with an amino acid having a nonpolar, aliphatic R group.
EE8. The isolated or purified polynucleotide of EE7, wherein Y185 is substituted with an amino acid sequence selected from glycine, alanine, valine, leucine, methionine, or isoleucine.
EE9. The isolated or purified polynucleotide of EE8, wherein Y185 is substituted with leucine (Y185L) [SEQ ID NO: 6].
EE10. A construct or vector comprising the isolated or purified polynucleotide of any one of EE1-EE3, operably linked to a promoter.
EE11. A construct or vector comprising the isolated or purified polynucleotide of any one of EE4-EE6, operably linked to a promoter.
EE12. A construct or vector comprising the isolated or purified polynucleotide of any one of EE7-EE9, operably linked to a promoter.
EE13. A host cell comprising and expressing the construct or vector of EE10.
EE14. The host cell of EE13, wherein the host cell is a Cyanobacterium.
EE15. A host cell comprising and expressing the construct or vector of EE11.
EE16. The host cell of EE15, wherein the host cell is a Cyanobacterium.
EE17. A host cell comprising and expressing the construct or vector of EE12.
EE18. The host cell of EE17, wherein the host cell is a Cyanobacterium.
EE19. A host cell comprising and expressing the isolated or purified polynucleotide of EE3 and further comprising and expressing the isolated or purified nucleic molecule of EE6.
EE20. The host cell of EE29, which further comprises and expresses the isolated or purified polynucleotide of EE9.
EE21. The host cell of EE19 or EE20, wherein the host cell is a Cyanobacterium.
EE22. An isolated or purified polypeptide having the amino acid sequence of SEQ ID NO: 4.
EE23. An isolated or purified polypeptide having the amino acid sequence of SEQ ID NO: 5.
EE24. An isolated or purified polypeptide having the amino acid sequence of SEQ ID NO: 6.
EE25. A method of producing phenylalanine in a prokaryotic host, which method comprises culturing the host cell of any one of EE13-EE21 in medium under culture conditions that support the production of phenylalanine, whereupon phenylalanine in the prokaryotic host is produced.
EE26. The method of EE25, which further comprises isolating the produced phenylalanine, e.g., by collecting phenylalanine from a biomass of the cultured prokaryotic host or a culture medium thereof.
EE27. The method of EE25 or EE26, wherein the prokaryotic host is Cyanobacterium, and the medium is modified BG-11.
EE28. The method of EE27, wherein the medium is supplemented with concentrated modified BG-11 medium about every three days.

### REFERENCES

1. Knoot, C. J., Ungerer, J., Wangikar, P. P. & Pakrasi, H. B. Cyanobacteria: Promising biocatalysts for sustainable chemical production. J. Biol. Chem. 293, 5044-5052 (2018).
2. Huang, Q., Jiang, E, Wang, L. & Yang, C. Design of Photobioreactors for Mass Cultivation of Photosynthetic Organisms. Engineering 3, 318-329 (2017).
3. Jaiswal, D., Sahasrabuddhe, D. & Wangikar, P. P. Cyanobacteria as cell factories: the roles of host and pathway engineering and translational research. Curr Opin. Biotechnol. 73, 314-322 (2022).
4. Whitney, S. M., Houtz, R. L. & Alonso, H. Advancing Our Understanding and Capacity to Engineer Nature's C02-Sequestering Enzyme, Rubisco. Plant Physiol. 155, 27-35 (2011).
5. Liang, E, Lindberg, P. & Lindblad, P. Engineering photoautotrophic carbon fixation for enhanced growth and productivity. Sustain. Energy Fuels 2, 2583-2600 (2018).
6. Kirst, H., Formighieri, C. & Melis, A. Maximizing photosynthetic efficiency and culture productivity in cyanobacteria upon minimizing the phycobilisome light-harvesting antenna size. Biochim. Biophys. Acta BBA—Bioenerg. 1837, 1653-1664 (2014).
7. Shen, B.-R. et al. Engineering a New Chloroplastic Photorespiratory Bypass to Increase Photosynthetic Efficiency and Productivity in Rice. Mol. Plant 12, 199-214 (2019).
8. Xu, H. et al. A synthetic light-inducible photorespiratory bypass enhances photosynthesis to improve rice growth and grain yield. Plant Commun. 4, 100641 (2023).
9. Kromdijk, J. et al. Improving photosynthesis and crop productivity by accelerating recovery from photoprotection. Science 354, 857-861 (2016).
10. Zhang, A., Carroll, A. L. & Atsumi, S. Carbon recycling by cyanobacteria: improving C02 fixation through chemical production. FEMS Microbiol. Lett. 364, (2017).
11. Abramson, B. W., Kachel, B., Kramer, D. M. & Ducat, D. C. Increased Photochemical Efficiency in Cyanobacteria via an Engineered Sucrose Sink. Plant Cell Physiol. 57, 2451-2460 (2016).
12. Santos-Merino, M. et al. Improved photosynthetic capacity and photosystem I oxidation via heterologous metabolism engineering in cyanobacteria. Proc. Natl. Acad. Sci. 118, e2021523118 (2021).
13. Oliver, J. W. K. & Atsumi, S. A carbon sink pathway increases carbon productivity in cyanobacteria. Metab. Eng. 29, 106-112 (2015).
14. Ni, J., Liu, H., Tao, E, Wu, Y. & Xu, P. Remodeling of the Photosynthetic Chain Promotes Direct CO₂ Conversion into Valuable Aromatic Compounds. Angew. Chem. Int. Ed. 57, 15990-15994 (2018).
15. Savakis, P. et al. Photosynthetic production of glycerol by a recombinant cyanobacterium. J. Biotechnol. 195, 46-51 (2015).
16. Kukil, K., Englund, E., Crang, N., Hudson, E. P. & Lindberg, P. Laboratory evolution of Synechocystis sp. PCC 6803 for phenylpropanoid production. Metab. Eng. 79, 27-37 (2023).
17. Ungerer, J. et al. Sustained photosynthetic conversion of C02 to ethylene in recombinant cyanobacterium Synechocystis 6803. Energy Environ. Sci. 5, 8998 (2012).
18. Grund, M., Jakob, T., Wilhelm, C., Buhler, B. & Schmid, A. Electron balancing under different sink conditions reveals positive effects on photon efficiency and metabolic activity of Synechocystis sp. PCC 6803. Biotechnol. Biofuels 12, 43 (2019).
19. Kramer, D. M. & Evans, J. R. The Importance of Energy Balance in Improving Photosynthetic Productivity. Plant Physiol. 155, 70-78 (2011).
20. Erdrich, P., Knoop, H., Steuer, R. & Klamt, S. Cyanobacterial biofuels: new insights and strain design strategies revealed by computational modeling. Microb. Cell Factories 13, 128 (2014).
21. Steichen, S. et al. Central transcriptional regulator controls photosynthetic growth and carbon storage in response to high light. Nat. Commun. 15, 4842 (2024).
22. Pogoryelov, D. et al. The oligomeric state of c rings from cyanobacterial F-ATP synthases varies from 13 to 15. J. Bacteriol. 189, 5895-5902 (2007).
23. Noctor, G. Review article. A re-evaluation of the ATP:NADPH budget during C3 photosynthesis: a contribution from nitrate assimilation and its associated respiratory activity? J. Exp. Bot. 49, 1895-1908 (1998).
24. Nogales, J., Gudmundsson, S., Knight, E. M., Palsson, B. O. & Thiele, I. Detailing the optimality of photosynthesis in cyanobacteria through systems biology analysis. Proc. Natl. Acad. Sci. 109, 2678-2683 (2012).
25. Allen, J. F. Cyclic, pseudocyclic and noncyclic photophosphorylation: new links in the chain. Trends Plant Sci. 8, 15-19 (2003).
26. Jaiswal, D. et al. Genome Features and Biochemical Characteristics of a Robust, Fast Growing and Naturally Transformable Cyanobacterium Synechococcus elongatus PCC 11801 Isolated from India. Sci. Rep. 8, 16632 (2018).
27. Stanier, R. Y., Deruelles, J., Rippka, R., Herdman, M. & Waterbury, J. B. Generic Assignments, Strain Histories and Properties of Pure Cultures of Cyanobacteria. Microbiology 111, 1-61 (1979).
28. Deshpande, A., Vue, J. & Morgan, J. Combining Random Mutagenesis and Metabolic Engineering for Enhanced Tryptophan Production in Synechocystis sp. Strain PCC 6803. Appl. Environ. Microbiol. 86, e02816-19 (2020).
29. Wick, R. R., Judd, L. M., Gorrie, C. L. & Holt, K. E. Unicycler: Resolving bacterial genome assemblies from short and long sequencing reads. PLoS Comput. Biol. 13, e1005595 (2017).
30. McKenna, A. et al. The Genome Analysis Toolkit: A MapReduce framework for analyzing next-generation DNA sequencing data. Genome Res. 20, 1297-1303 (2010).
31. States, D. J. & Gish, W. QGB: Combined Use of Sequence Similarity and Codon Bias for Coding Region Identification. J. Comput. Biol. 1, 39-50 (1994).
32. Ritchie, R. J. Consistent Sets of Spectrophotometric Chlorophyll Equations for Acetone, Methanol and Ethanol Solvents. Photosynth. Res. 89, 27-41 (2006).
33. Porra, R. J., Thompson, W. A. & Kriedemann, P. E. Determination of accurate extinction coefficients and simultaneous equations for assaying chlorophylls a and b extracted with four different solvents: verification of the concentration of chlorophyll standards by atomic absorption spectroscopy. Biochim. Biophys. Acta BBA—Bioenerg. 975, 384-394 (1989).
34. Campbell, D., Hurry, V., Clarke, A. K., Gustafsson, P. & Oquist, G. Chlorophyll Fluorescence Analysis of Cyanobacterial Photosynthesis and Acclimation. Microbiol. Mol. Biol. Rev. 62, 667-683 (1998).
35. Genty, B., Briantais, J.-M. & Baker, N. R. The relationship between the quantum yield of photosynthetic electron transport and quenching of chlorophyll fluorescence. Biochim. Biophys. Acta BBA—Gen. Subj. 990, 87-92 (1989).
36. Ralph, P. J. & Gademann, R. Rapid light curves: A powerful tool to assess photosynthetic activity. Aquat. Bot. 82, 222-237 (2005).
37. Baker, N. R. Chlorophyll Fluorescence: A Probe of Photosynthesis In Vivo. Annu. Rev. Plant Biol. 59, 89-113 (2008).
38. Vega de Luna, F., Córdoba-Granados, J. J., Dang, K.-V., Roberty, S. & Cardol, P. In vivo assessment of mitochondrial respiratory alternative oxidase activity and cyclic electron flow around photosystem I on small coral fragments. Sci. Rep. 10, 17514 (2020).
39. Klughammer, C. & Schreiber, U. An improved method, using saturating light pulses, for the determination of photosystem I quantum yield via P700+-absorbance changes at 830 un. Planta 192, 261-268 (1994).
40. Berla, B. M., Saha, R., Maranas, C. D. & Pakrasi, H. B. Cyanobacterial Alkanes Modulate Photosynthetic Cyclic Electron Flow to Assist Growth under Cold Stress. Sci. Rep. 5, 14894 (2015).
41. Holland, S. C. et al. Impacts of genetically engineered alterations in carbon sink pathways on photosynthetic performance. Algal Res. 20, 87-99 (2016).
42. Hall, G. C. & Jensen, R. A. Enzymological Basis for Growth Inhibition by L-Phenylalanine in the Cyanobacterium Synechocystis sp. 29108. J. Bacteriol. 144, 1034-1042 (1980).
43. Brey, L. F. et al. Metabolic engineering of Synechocystis sp. PCC 6803 for the production of aromatic amino acids and derived phenylpropanoids. Metab. Eng. 57, 129-139 (2020).
44. Grant, G. A. The ACT Domain: A Small Molecule Binding Domain and Its Role as a Common Regulatory Element. J. Biol. Chem. 281, 33825-33829 (2006).
45. Vue, Jeremiah. Metabolic Engineering for Aromatic Amino Acids in Cyanobacteria. (Purdue University, West Lafayette, IN, 2017).
46. Wiriyathanawudhiwong, N., Ohtsu, I., Li, Z.-D., Mori, H. & Takagi, H. The outer membrane TolC is involved in cysteine tolerance and overproduction in Escherichia coli. Appl. Microbiol. Biotechnol. 81, 903-913 (2009).
47. Dvornyk, V. & Mei, Q. Evolution of kaiA, a key circadian gene of cyanobacteria. Sci. Rep. 11, 9995 (2021).
48. van Alphen, P., Abedini Najafabadi, H., Branco dos Santos, F. & Hellingwerf, K. J. Increasing the Photoautotrophic Growth Rate of Synechocystis sp. PCC 6803 by Identifying the Limitations of Its Cultivation. Biotechnol. J. 13, 1700764 (2018).
49. Wlodarczyk, A., Selio, T. T., Norling, B. & Nixon, P. J. Newly discovered Synechococcus sp. PCC 11901 is a robust cyanobacterial strain for high biomass production. Commun. Biol. 3, 215 (2020).
50. Stamatakis, K., Tsimilli-Michael, M. & Papageorgiou, G. C. On the question of the light-harvesting role of β-carotene in photosystem II and photosystem I core complexes. Plant Physiol. Biochem. 81, 121-127 (2014).
51. Sozer, O. et al. Involvement of Carotenoids in the Synthesis and Assembly of Protein Subunits of Photosynthetic Reaction Centers of Synechocystis sp. PCC 6803. Plant Cell Physiol. 51, 823-835 (2010).
52. Schafer, L., Vioque, A. & Sandmann, G. Functional in situ evaluation of photosynthesis-protecting carotenoids in mutants of the cyanobacterium Synechocystis PCC6803. J. Photochem. Photobiol. B 78, 195-201 (2005).
53. Santos-Merino, M., Singh, A. K. & Ducat, D. C. Sink Engineering in Photosynthetic Microbes. in Cyanobacteria Biotechnology (eds. Nielsen, J., Lee, S., Stephanopoulos, G. & Hudson, P.) 171-209 (Wiley, 2021). doi:10.1002/9783527824908.ch6.
54. Liu, Y. et al. Genetic engineering of Escherichia coli to improve L-phenylalanine production. BMC Biotechnol. 18, 5 (2018).
55. Brandenburg, F. et al. Trans-4-hydroxy-L-proline production by the cyanobacterium Synechocystis sp. PCC 6803. Metab. Eng. Commun. 12, e00155 (2021).
56. Masojídek, J. & Torzillo, G. Mass Cultivation of Freshwater Microalgae☆. in Reference Module in Earth Systems and Environmental Sciences B9780124095489094000 (Elsevier, 2014). doi:10.1016/B978-0-12-409548-9.09373-8.
57. Chan, M.-S. & Hsu, W.-H. Cloning ofm-Fluorophenylalanine-Resistant Gene and Mutational Analysis of Feedback-Resistant Prephenate Dehydratase from Corynebacterium glutamicum. Biochem. Biophys. Res. Commun. 219, 537-542 (1996).
58. Mellor, S. B. et al. Defining optimal electron transfer partners for light-driven cytochrome P450 reactions. Metab. Eng. 55, 33-43 (2019).
59. Shastri, A. A. & Morgan, J. A. Flux Balance Analysis of Photoautotrophic Metabolism. Biotechnol. Prog. 21, 1617-1626 (2005).
60. Montesinos, M. L., Herrero, A. & Flores, E. Amino acid transport in taxonomically diverse cyanobacteria and identification of two genes encoding elements of a neutral amino acid permease putatively involved in recapture of leaked hydrophobic amino acids. J. Bacteriol. 179, 853-862 (1997).
61. Huang, W., Yang, Y.-J., Hu, H. & Zhang, S.-B. Different roles of cyclic electron flow around photosystem I under sub-saturating and saturating light intensities in tobacco leaves. Front. Plant Sci. 6, (2015).

The term “about” as used herein can allow for a degree of variability in a value or range, for example, within 10%, within 5%, or within 1% of a stated value or of a stated limit of a range.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section.

Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

All publications and patents mentioned herein are incorporated by reference in their entireties for all purposes. In the event of inconsistent usages between this document and those documents so incorporated by reference, the usage in the incorporated reference should be considered supplementary to that of this document; for irreconcilable inconsistencies, the usage in this document controls.

What is claimed is:

1. An isolated or purified polynucleotide encoding a prephenate dehydratase having an amino acid sequence that is at least about 90% identical to the amino acid sequence of SEQ ID NO: 1:
1 MAALRFQAWL TDQDQQPSHL LACRSIPATL QTLADGTVDY AVVPVENSVE GSVAATLDSL
61 WQLPQLSIQR ALILPIAHAL ISFEDDRTAI RQVLSHPQAL AQCQQWLQRQ LPQAELIPTN
121 STTEALQDLE RHPQRAVIAS TRAAELYQMP IQSFPINDSP DNRTRFWVVS RNLTPGGACT
181 SLSFSLDANV PGALVKPLQI LAERQINLSR IESRPTKRSL [symbol] EYLFFLDLE ADLREPAIAQ
241 AVQAVADCTE QLRVLGSYDS LDFTQVVQPS [SEQ ID NO: 1], in which the glycine at amino acid position 221 (G221) is substituted with an amino acid having an aromatic R group.

2. The isolated or purified polynucleotide of claim 1, wherein G221 is substituted with an amino acid selected from phenylalanine, tyrosine, and tryptophan.

3. The isolated or purified polynucleotide of claim 2, wherein G221 is substituted with tryptophan (G221W) [SEQ ID NO: 4].

4. An isolated or purified polynucleotide encoding a TolC family protein having an amino acid sequence that is at least about 90% identical to the amino acid sequence of SEQ ID NO: 2:
1 MAAFLYRLSL LSALAIAAHG MEPPAAIADI VDPQATGPSP TIAQNSPPPA ATTPAPTTPP
61 SSPVKEVVPD ANLLKELQAN PNPFQLPNQP NQVQTESLQP LTLEQALNLA RLNNPQVQIR
121 QLQVEQRRAA LRGTEAALYP TLGLQGNAGY QQSGNRLNVT EGSTVQPTGS SLFTTLGQSS
181 IGATLNLNYT IFDFVRGAQL AASRDQVTQA ELDLEATLED LQLTVSEAYY QLQNADQLVR
241 IARESVVASE RSLKDAEALF RAGVGTQFDV LRQQVQLAQD QQNLVDSIGN QDKARRSLVQ
301 ALNLPQNVNV LTADPVELAA PWNLSLDESI VLAFQNRPEL EREVLQRNIS YNQAQAARGQ
361 ILPQLGLQAS YGVTGSINSN LRSGSQALTF PSPTLTNNSN YSYSIGLVLN VPLFDGGLAN
421 ANAQQQELNG QIAEQNFVLT RNQIRTDVET AFYDLQTNLA NIGTTRKAVE QAREALRLAR
481 LRFQAGVGTQ TEVIDSQRDL TRAEANALNA ITAYNLALAR IKRAVSNVSN [symbol] ARVGS [SEQ ID NO: 2], in which the leucine at amino acid position 531 (L531) is substituted with an amino acid having an aromatic R group.

5. The isolated or purified polynucleotide of claim 4, wherein L531 is substituted with an amino acid selected from phenylalanine, tyrosine, and tryptophan.

6. The isolated or purified polynucleotide of claim 5, wherein L531 is substituted with tryptophan (L531W) [SEQ ID NO: 5].

7. An isolated or purified polynucleotide encoding a circadian clock protein having an amino acid sequence that is at least about 90% identical to the amino acid sequence of SEQ ID NO: 3:
1 MLSQIAICIW VESTAILQDC QQALAGDRYQ LQICSSGDLL LDYAQTHRDQ IDCLLLVATN
61 PGCKTVIQQL CFQGIVVPAI VVGDRDTDDV TDTQKDWVYH SAELHLGIHQ LEQLPYQVDA
121 ALAEFLRQAP VETIADQVML MAATHDPELA SHQRDLAQRL QERLGYLGVY YKRDPDRFLR
181 NLPA [symbol] EGQKL LEAMQTSYRE IVLSYFSPNS NLNQSLDNFV NMAFFADVPV TQVVEIHMEL
241 MDEFAKKLRV EGRSEDILLD YRLTLIDVIA HLCEMYRRSI PRET [SEQ ID NO: 3], in which there is a frameshift mutation at tyrosine at amino acid position 185 (Y185) resulting in the substitution of Y185 with an amino acid having a nonpolar, aliphatic R group.

8. The isolated or purified polynucleotide of claim 7, wherein Y185 is substituted with an amino acid sequence selected from glycine, alanine, valine, leucine, methionine, or isoleucine.

9. The isolated or purified polynucleotide of claim 8, wherein Y185 is substituted with leucine (Y185L) [SEQ ID NO: 6].

10. A construct or vector comprising the isolated or purified polynucleotide of claim 1, operably linked to a promoter.

11. A construct or vector comprising the isolated or purified polynucleotide of claim 4, operably linked to a promoter.

12. A construct or vector comprising the isolated or purified polynucleotide of claim 7, operably linked to a promoter.

13. A host cell comprising and expressing the construct or vector of claim 10.

14. The host cell of claim 13, wherein the host cell is a Cyanobacterium.

15. A host cell comprising and expressing the construct or vector of claim 11.

16. The host cell of claim 15, wherein the host cell is a Cyanobacterium.

17. A host cell comprising and expressing the construct or vector of claim 12.

18. The host cell of claim 17, wherein the host cell is a Cyanobacterium.

19. A host cell comprising and expressing an isolated or purified polynucleotide encoding a prephenate dehydratase having an amino acid sequence that is at least about 90% identical to the amino acid sequence of SEQ ID NO: 4, an isolated or purified polynucleotide encoding a TolC family protein having an amino acid sequence that is at least about 90% identical to the amino acid sequence of SEQ ID NO: 5.

20. The host cell of claim 19, which further comprises and expresses an isolated or purified polynucleotide encoding an isolated or purified polynucleotide encoding a circadian clock protein having an amino acid sequence that is at least about 90% identical to the amino acid sequence of SEQ ID NO: 6.

21. The host cell of claim 19, wherein the host cell is a Cyanobacterium.

22. The host cell of claim 20, wherein the host cell is a Cyanobacterium.

23. An isolated or purified polypeptide having the amino acid sequence of SEQ ID NO: 4.

24. An isolated or purified polypeptide having the amino acid sequence of SEQ ID NO: 5.

25. An isolated or purified polypeptide having the amino acid sequence of SEQ ID NO: 6.

26. A method of producing phenylalanine in a prokaryotic host, which method comprises culturing a host cell in medium under culture conditions that support the production of phenylalanine, wherein the host cell comprises and expresses a construct or vector comprising:
(a) an isolated or purified polynucleotide encoding a prephenate dehydratase having an amino acid sequence that is at least about 90% identical to the amino acid sequence of SEQ ID NO: 1, in which the glycine at amino acid position 221 (G221) is substituted with an amino acid having an aromatic R group, operably linked to a promoter;
(b) an isolated or purified polynucleotide encoding a TolC family protein having an amino acid sequence that is at least about 90% identical to the amino acid sequence of SEQ ID NO: 2, in which the leucine at amino acid position 531 (L531) is substituted with an amino acid having an aromatic R group, operably linked to a promoter;
(c) an isolated or purified polynucleotide encoding a circadian clock protein having an amino acid sequence that is at least about 90% identical to the amino acid sequence of SEQ ID NO: 3, in which there is a frameshift mutation at tyrosine at amino acid position 185 (Y185) resulting in the substitution of Y185 with an amino acid having a nonpolar, aliphatic R group, operably linked to a promoter;
(d) an isolated or purified polynucleotide encoding a prephenate dehydratase having an amino acid sequence that is at least about 90% identical to the amino acid sequence of SEQ ID NO: 4, an isolated or purified polynucleotide encoding a TolC family protein having an amino acid sequence that is at least about 90% identical to the amino acid sequence of SEQ ID NO: 5, operably linked to a promoter; or
(e) an isolated or purified polynucleotide encoding a prephenate dehydratase having an amino acid sequence that is at least about 90% identical to the amino acid sequence of SEQ ID NO: 4, an isolated or purified polynucleotide encoding a TolC family protein having an amino acid sequence that is at least about 90% identical to the amino acid sequence of SEQ ID NO: 5, and an isolated or purified polynucleotide encoding an isolated or purified polynucleotide encoding a circadian clock protein having an amino acid sequence that is at least about 90% identical to the amino acid sequence of SEQ ID NO: 6, operably linked to a promoter;
whereupon phenylalanine in the prokaryotic host is produced.

27. The method of claim 26, which further comprises isolating the produced phenylalanine.

28. The method of claim 26, wherein the prokaryotic host is Cyanobacterium, and the medium is modified BG-11.

29. The method of claim 28, wherein the medium is supplemented with concentrated modified BG-11 medium about every three days.


SEQUENCE LISTING (6 sequences)

SEQ ID NO: 1 - protein, 270 aa, synthetic construct
MAALRFQAWLTDQDQQPSHLLACRSIPATLQTLADGTVDYAVVPVENSVEGSVAATLDSLWQLPQLSIQRALILPIAHALISFEDDRTAIRQVLSHPQALAQCQQWLQRQLPQAELIPTNSTTEALQDLERHPQRAVIASTRAAELYQMPIQSFPINDSPDNRTRFWVVSRNLTPGGACTSLSFSLDANVPGALVKPLQILAERQINLSRIESRPTKRSLGEYLFFLDLEADLREPAIAQAVQAVADCTEQLRVLGSYDSLDFTQVVQPS

SEQ ID NO: 2 - protein, 536 aa, synthetic construct
MAAFLYRLSLLSALAIAAHGMEPPAAIADIVDPQATGPSPTIAQNSPPPAATTPAPTTPPSSPVKEVVPDANLLKELQANPNPFQLPNQPNQVQTESLQPLTLEQALNLARLNNPQVQIRQLQVEQRRAALRGTEAALYPTLGLQGNAGYQQSGNRLNVTEGSTVQPTGSSLFTTLGQSSIGATLNLNYTIFDFVRGAQLAASRDQVTQAELDLEATLEDLQLTVSEAYYQLQNADQLVRIARESVVASERSLKDAEALFRAGVGTQFDVLRQQVQLAQDQQNLVDSIGNQDKARRSLVQALNLPQNVNVLTADPVELAAPWNLSLDESIVLAFQNRPELEREVLQRNISYNQAQAARGQILPQLGLQASYGVTGSINSNLRSGSQALTFPSPTLTNNSNYSYSIGLVLNVPLFDGGLANANAQQQELNGQIAEQNFVLTRNQIRTDVETAFYDLQTNLANIGTTRKAVEQAREALRLARLRFQAGVGTQTEVIDSQRDLTRAEANALNAITAYNLALARIKRAVSNVSNLARVGS

SEQ ID NO: 3 - protein, 284 aa, synthetic construct
MLSQIAICIWVESTAILQDCQQALAGDRYQLQICSSGDLLLDYAQTHRDQIDCLLLVATNPGCKTVIQQLCFQGIVVPAIVVGDRDTDDVTDTQKDWVYHSAELHLGIHQLEQLPYQVDAALAEFLRQAPVETIADQVMLMAATHDPELASHQRDLAQRLQERLGYLGVYYKRDPDRFLRNLPAYEGQKLLEAMQTSYREIVLSYFSPNSNLNQSLDNFVNMAFFADVPVTQVVEIHMELMDEFAKKLRVEGRSEDILLDYRLTLIDVIAHLCEMYRRSIPRET

SEQ ID NO: 4 - protein, 270 aa, Synthetic construct
MAALRFQAWLTDQDQQPSHLLACRSIPATLQTLADGTVDYAVVPVENSVEGSVAATLDSLWQLPQLSIQRALILPIAHALISFEDDRTAIRQVLSHPQALAQCQQWLQRQLPQAELIPTNSTTEALQDLERHPQRAVIASTRAAELYQMPIQSFPINDSPDNRTRFWVVSRNLTPGGACTSLSFSLDANVPGALVKPLQILAERQINLSRIESRPTKRSLWEYLFFLDLEADLREPAIAQAVQAVADCTEQLRVLGSYDSLDFTQVVQPS

SEQ ID NO: 5 - protein, 536 aa, synthetic construct
MAAFLYRLSLLSALAIAAHGMEPPAAIADIVDPQATGPSPTIAQNSPPPAATTPAPTTPPSSPVKEVVPDANLLKELQANPNPFQLPNQPNQVQTESLQPLTLEQALNLARLNNPQVQIRQLQVEQRRAALRGTEAALYPTLGLQGNAGYQQSGNRLNVTEGSTVQPTGSSLFTTLGQSSIGATLNLNYTIFDFVRGAQLAASRDQVTQAELDLEATLEDLQLTVSEAYYQLQNADQLVRIARESVVASERSLKDAEALFRAGVGTQFDVLRQQVQLAQDQQNLVDSIGNQDKARRSLVQALNLPQNVNVLTADPVELAAPWNLSLDESIVLAFQNRPELEREVLQRNISYNQAQAARGQILPQLGLQASYGVTGSINSNLRSGSQALTFPSPTLTNNSNYSYSIGLVLNVPLFDGGLANANAQQQELNGQIAEQNFVLTRNQIRTDVETAFYDLQTNLANIGTTRKAVEQAREALRLARLRFQAGVGTQTEVIDSQRDLTRAEANALNAITAYNLALARIKRAVSNVSNWARVGS

SEQ ID NO: 6 - protein, 284 aa, synthetic construct
MLSQIAICIWVESTAILQDCQQALAGDRYQLQICSSGDLLLDYAQTHRDQIDCLLLVATNPGCKTVIQQLCFQGIVVPAIVVGDRDTDDVTDTQKDWVYHSAELHLGIHQLEQLPYQVDAALAEFLRQAPVETIADQVMLMAATHDPELASHQRDLAQRLQERLGYLGVYYKRDPDRFLRNLPALEGQKLLEAMQTSYREIVLSYFSPNSNLNQSLDNFVNMAFFADVPVTQVVEIHMELMDEFAKKLRVEGRSEDILLDYRLTLIDVIAHLCEMYRRSIPRET